(19) United States
(12) Patent Application Publication
(10) Pub. No.: US 2026/0345853 A1
(43) Pub. Date: Nov. 5, 2026

(54) FAR-UV APPARATUS FOR INACTIVATING PATHOGENS IN AIR

(71) Applicant: The OSLUV Project, St. Paul, MN (US)

(72) Inventors: Aaron Collins, Minneapolis, MN (US); Chia Hua M. Pang, St. Paul, MN (US); Louis Goessling, Minneapolis, MN (US); Christopher Richard Saline Libby, St. Louis Park, MN (US); Rachel Andreasen, St. Paul, MN (US)

(21) Appl. No.: 19/486,377

(22) PCT Filed: May 22, 2024
(86) PCT No.: PCT/US2024/030579

§ 371 Date: Nov. 21, 2025

Related U.S. Application Data
(60) Provisional application No. 63/468,255, filed on May 22, 2023.

(51) Int. Cl. A61L 9/20; B01D 46/00

(52) U.S. Cl. CPC A61L 9/20; B01D 46/0028; B01D 46/0045; A61L 2209/12; A61L 2209/14; A61L 2209/16; B01D 2273/30; B01D 2279/50; B01D 2279/65

(57) ABSTRACT

An apparatus for inactivating a pathogen in air. The apparatus has an air stream housing integrated with a far-UV radiation source. The air stream housing includes a mechanical filter, a scrubber, or both, and a fan system provides an air stream through the air stream housing. The far-UV radiation source is integrated with the air stream unit external to the air flow chamber and adjacent to at least one of the air intake or air exhaust, and the far-UV radiation source is positioned to direct radiation away from the air stream housing. A system and method for inactivating a pathogen in air are also described.

### CROSS-REFERENCE TO RELATED APPLICATIONS

This application claims the benefit of priority to U.S. Provisional Patent Application Ser. No. 63/468,255, filed May 22, 2023, the disclosure of which is incorporated herein in its entirety by reference.

### TECHNICAL FIELD

This document pertains generally, but not by way of limitation, to apparatus, systems, and methods for inactivating pathogens in air. More specifically, but not by way of limitation, the present application relates to use of far-UV light, including 222 nm light, to inactivate pathogens in air.

### BACKGROUND

The general use of ultraviolet (UV) light has long been recognized as a mechanism to disinfect surfaces and indoor air. In the early 1940s, Wells et al. published a seminal work utilizing germicidal lamps to study disinfection of air in schools. (Wells et al., Am J Hyg. 1942; 35:97-121). More recent innovations utilize filtered, quasi-monochromatic short wavelength germicidal ultraviolet (GUV) radiation, particularly 222 nm light, which can be provided by KrCl excimer lamps. Filtered GUV devices that eliminate harmful wavelengths have been reported as safe, yet continued innovations are necessary to improve safety and increase efficacy of GUV devices.

### SUMMARY

The present disclosure provides an apparatus for inactivating a pathogen in air, comprising an air stream housing defining an air flow chamber, an air intake, and an air exhaust; a filter system comprising a mechanical filter, a scrubber, or both, disposed in the air flow chamber; a fan system configured to provide an air stream through the air intake, air flow chamber, and air exhaust; and a far-UV radiation source that emits radiation comprising one or more wavelengths of about 210 nm to about 230 nm, wherein the far-UV radiation source is integrated with the air stream unit external to the air flow chamber and adjacent to at least one of the air intake or air exhaust, and the far-UV radiation source is positioned to direct radiation away from the air stream housing, filter, and scrubber, if present. The disclosure also provides a portable kit for inactivating a pathogen in air, comprising such apparatus.

The present disclosure further provides a system for inactivating a pathogen in air, comprising: a far-UV radiation source configured for irradiating air of a human-use space, wherein the far-UV radiation source emits radiation comprising one or more wavelengths of about 210 nm to about 230 nm; an air stream housing that defines an air flow chamber, an air intake, and an air exhaust; a fan system configured to displace irradiated air proximate to the radiation source and provide an air stream through the air intake, air flow chamber, and air exhaust; optionally, a filter disposed in the air flow chamber configured for removing particles from the air; and optionally, a scrubber disposed in the air flow chamber configured for removing reactive agents from the air; wherein the far-UV radiation source is integrated with the air stream unit external to the air flow chamber and adjacent to at least one of the air intake or air exhaust, and the far-UV radiation source is positioned such that substantially all radiation is directed away from the air stream housing.

The present disclosure yet further provides a method for inactivating a pathogen in air, comprising: irradiating air in a human-use space with radiation from a far-UV radiation source, wherein the radiation is configured to inactivate a pathogen in air and comprises one or more wavelengths of about 210 nm to about 230 nm; generating an air stream through an air stream housing and displacing irradiated air proximate to the far-UV radiation source using a fan system, wherein the air stream housing defines an air flow chamber, an air intake, and an air exhaust; optionally, removing particles from the air stream in the air stream housing; and optionally, removing reactive agents from the air stream in the air stream housing; wherein the far-UV radiation source is integrated with the air stream unit external to the air flow chamber and adjacent to at least one of the air intake or air exhaust, and the far-UV radiation source is positioned such that substantially all radiation is directed away from the air stream housing.

### BRIEF DESCRIPTION OF THE DRAWINGS

FIG. 1 is a schematic of a far-UV apparatus having a radiation source integrated with an air stream apparatus.

FIG. 2 is a schematic of various examples of a far-UV apparatus having a radiation source integrated with an air stream apparatus. FIG. 2A illustrates two configuration with front-back air flow and one configuration with front side air flow. FIG. 2B illustrates a configuration with front top air flow and a configuration with front-front air flow.

FIG. 3 is a schematic of various further examples of a far-UV apparatus having a radiation source integrated with an air stream apparatus. The various illustrated examples utilize one or more far-UV radiation source externally integrated with one or more air stream unit.

FIG. 4 is a schematic of yet further examples of a far-UV apparatus having a radiation source integrated with an air stream apparatus. The air stream is configured to follow the edge of the broadcast pattern of the far-UV radiation source. FIG. 4A illustrates two configurations where an air stream unit is positioned below the far-UV radiation source, and may be attached via a fastener, hinge, reversible coupling, or other connection permitting degrees of freedom of movement, removability, or articulation. FIG. 4B illustrates a configuration with air flow units above and below the far-UV radiation source and a configuration with a far-UV radiation source at an angle directed through the area where an air stream is generated.

FIG. 5 is a front view of various examples of a far-UV apparatus having a radiation source integrated with an air stream apparatus. FIG. 5A illustrates configurations with one or more air intakes (or air exhausts) on the front face of the apparatus below the far-UV radiation source, which can feed to one or more air stream unit. FIG. 5B illustrates configurations with two air intakes (or air exhausts) on the front face of the apparatus, which can feed to one or more air stream unit.

FIG. 6 is a schematic of an example of a far-UV apparatus having a radiation source integrated with an air stream apparatus. FIG. 6 shows a section view and a front view.

FIG. 7 is a schematic of an example of a far-UV apparatus having a radiation source integrated with an air stream apparatus. FIG. 7A shows a section view and a front view. FIG. 7B shows a top view and a side view of the integrated apparatus mounted on a telescoping stand.

FIG. 8 is a schematic of an example of a far-UV apparatus having a radiation source integrated with an air stream apparatus. FIG. 8 shows an unmounted section view, a section view mounted on a telescoping stand, and a front view.

FIG. 9 is a schematic of an example of a far-UV apparatus having a radiation source integrated with an air stream apparatus. FIG. 9 shows a section view, a front view, and a top view.

FIG. 10 is a schematic of an example of a far-UV apparatus having a radiation source integrated with an air stream apparatus. FIG. 10 shows a section view, a front view, and a top view.

FIG. 11 is a schematic of an example of a far-UV apparatus having a radiation source integrated with an air stream apparatus. FIG. 11 shows a section view, a front view, and a top view.

FIG. 12 is a diagram illustrating an advantage of a far-UV radiation source integrated externally relative to an air stream unit, which is oriented to displace irradiated air proximate to the radiation source.

FIG. 13 provides a fluence rate contour plot illustrating regions of higher fluence rate provided by an example far-UV radiation source.

FIG. 14 provides an air velocity contour plot, generated via a fluid dynamics simulation model, illustrating air velocity in a space treated with a far-UV radiation source alone.

FIG. 15 provides an air velocity contour plot, generated via a fluid dynamics simulation model, illustrating air velocity in a human-use space treated with an apparatus having a far-UV radiation source and air stream unit with a HEPA filter, which provides an air stream in the area of irradiated air in a pull configuration that captures irradiated air.

FIG. 16 provides an air velocity contour plot, generated via a fluid dynamics simulation model, illustrating air velocity in a human-use space treated with an apparatus having a far-UV radiation source and air stream unit with a MERV13 filter, which provides an air stream in the area of irradiated air in a pull configuration that captures irradiated air.

FIG. 17 provides an air velocity contour plot, generated via a fluid dynamics simulation model, illustrating air velocity in a human-use space treated with an apparatus having a far-UV radiation source and air stream unit with a MERV11 filter, which provides an air stream in the area of irradiated air in a pull configuration that captures irradiated air.

FIG. 18 provides an air velocity contour plot, generated via a fluid dynamics simulation model, illustrating air velocity in a human-use space treated with an apparatus having a far-UV radiation source and air stream unit with a HEPA filter, which provides an air stream in the area of irradiated air in a push configuration that exhausts purified air to the area of irradiation.

FIG. 19 provides an air velocity contour plot, generated via a fluid dynamics simulation model, illustrating air velocity in a human-use space treated with an apparatus having a far-UV radiation source and air stream unit with a MERV13 filter, which provides an air stream in the area of irradiated air in a push configuration that exhausts purified air to the area of irradiation.

FIG. 20 provides an air velocity contour plot, generated via a fluid dynamics simulation model, illustrating air velocity in a human-use space treated with an apparatus having a far-UV radiation source and air stream unit with a MERV11 filter, which provides an air stream in the area of irradiated air in a push configuration that exhausts purified air to the area of irradiation.

FIG. 21 provides an air velocity contour plot, generated via a fluid dynamics simulation model, illustrating air velocity in a human-use space treated with a far-UV radiation source and a separate modeled source of an air stream located on the other side of the space.

FIG. 22 provides a concentration contour plot, generated via a fluid dynamics simulation model, illustrating concentration of a VOC in a human-use space treated with an apparatus having a far-UV radiation source and air stream unit, which provides an air stream in the area of irradiated air in a push configuration that exhausts purified air to the area of irradiation.

FIG. 23 provides a concentration contour plot, generated via a fluid dynamics simulation model, illustrating concentration of a VOC in a human-use space treated with an apparatus having a far-UV radiation source and air stream unit, which provides an air stream in the area of irradiated air in a pull configuration that captures irradiated air.

FIG. 24 illustrates how a far-UV radiation source can be integrated with an air stream unit to position a localized air stream in the areas of high radiation intensity.

### DETAILED DESCRIPTION

The present disclosure provides, among other things, an apparatus, system, and method for inactivating pathogens in air. The apparatus, system, and method involve use of a far-UV radiation source integrated with a housing that provides and directs an air stream relative to the radiation source. The air stream is generated by one or more fans integrated with the housing, which orients the air stream and provides air flow at irradiated air proximate to the radiation source. The far-UV radiation source is external to the housing and is configured to direct radiation away from the housing and irradiate external air that is outside of the housing. The apparatus, systems, and methods are useful for safely inactivating airborne pathogens in occupied space.

The generated air stream can displace air from the irradiated area near the radiation source. Irradiated air that is closer to the radiation source is subject to a higher intensity of radiation (e.g., a greater number of protons, higher fluence rate, or higher irradiance) per an inverse square relationship between radiation intensity and distance from the source. The positioning of the air stream housing relative to the far-UV radiation source can be configured to process the irradiated air near the radiation source. Filters and Scrubbers can be utilized within the housing to process the air, such as by removing particulate material or reactive components from the air. The air stream housing can further provide filtered and/or scrubbed air to the area of irradiation. The air stream housing can serve to mix and displace air in the area of irradiation.

The presently described apparatus, system, and method can provide a solution for several unappreciated problems in the art. For example, compared to approaches that provide irradiation in air ducts, HVAC, or filtration systems, various aspects described herein advantageously inactivate pathogens by decoupling the dwell time of airborne pathogens in the air subjected to far-UV radiation treatment from the removal of non-pathogenic airborne contaminants in the air stream by filtration and/or scrubbing. Surprisingly, the apparatus, systems, and methods described herein can benefit from filters and scrubbers having higher, rather than lower, penetration due to providing increased airflow near the radiation source and through the housing. Fast passage through a high penetration filter and/or scrubber and subsequent circulation through irradiated space provides advantages of multipass filtration and/or scrubbing, but also increased total irradiation time for pathogens in irradiated space in the event of multiple passes. The apparatus can treat a space without relying on ventilation, duct work, or an HVAC system. It can also offer advantages over ventilation or HVAC systems due to portability, multipass filtering and/or scrubbing, and continuous irradiation of the treatment space.

Additionally, the various apparatus, systems, and methods described herein can provide a solution for addressing potentially harmful airborne agents that can arise due to far-UV radiation of air, including those that arise from far-UV radiation having a wavelength of 222 nm or longer. Further, various apparatus, systems, and methods described herein can provide a solution for providing germicidal disinfection without generating harmful byproducts; for example, providing a localized air stream in an area of high UV radiation can reduce amount of radiation delivered to certain airborne particles while still achieving sufficient germicidal conditions for pathogens. A robust localized air stream can also have the advantage of providing further clearance by facilitating multiple passes through the system. Further, various apparatus, systems, and methods described herein can provide a solution for addressing airborne pathogens that occur around head level, e.g., four to six feet off the ground, due to breathing, talking, speaking, coughing, sneezing, and the like, which are associated with short range person-to-person transmission. The air stream provided can advantageously circulate air at and around the apparatus by moving air subjected to the higher intensities of far-UV radiation close to the far-UV radiation source. The air stream can also have the advantageous effect of moving air closest to the occupants, particularly so as to mix the air around head level with the rest of the air in the irradiated space. The air stream can be provided diffusely, so as to move air without partitioning the room into separate air streams, without generating an air wall that would reduce mixing between air containing pathogens and air that is already clean, and without causing discomfort or inconvenience to occupants. Increased airflow without partitioning the treated area into separate airspaces facilitates air exchange, filtration, and/or scrubbing of the entire treated space.

### DEFINITIONS

In this document, the terms “a”, “an”, or “the” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, apparatus, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about” as used herein can allow for a degree of variability in a value or range, for example, within 10%, within 5%, or within 1% of a stated value or of a stated limit of a range, and includes the exact stated value or range.

The term “substantially” as used herein, depending on context, can refer to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the term “pathogen” includes viruses, bacteria, infectious microbes, fungi, protozoa, parasites, and other agents, or components thereof, that are capable of causing disease or infection in humans or animals. Pathogens includes pathogens in droplets, pathogens in aerosols, and free pathogens in air.

As used herein, the term “inactivation” refers to a partial or complete reduction in a pathogens ability to cause disease or infection. Without intending to limit to any particular theory of mechanism, inactivation may be achieved by damaging or destroying the pathogen's genetic material, membrane, or other structures through exposure to far-UV radiation. In some contexts, inactivation can refer to inactivating a pathogens ability to reproduce, replicate, enter a cell, or evade the immune system. Inactivation of pathogens can be useful for disinfection or decontamination purposes.

As used herein, “human-use space” or “occupiable space” is an area that is designed or intended for use by humans. Examples include, but are not limited to, rooms, restaurants, schools, workplaces, offices, hospitals, cafeterias, event space, bars, theaters, gyms, retail space, public transportation vehicles, public transportation stations, airports, and airplanes. Human-use space or occupiable space can include indoor space and outdoor space. Human-use space or occupiable space can be empty, or it can be occupied space, for example by one or more occupants. Indoor space can include indoor space that is open, ventilated, or non-ventilated. In various examples, indoor space can have windows, or mechanical ventilation such as a HVAC system including air supplies and air returns. An occupiable space or occupied space can also refer to an animal-use space or plant-use space. The term “treatment space” refers to the occupiable space having the air that is irradiated to inactivate pathogens in air. As such, the volume of the treatment space refers to the volume of the entire human-use space, e.g., a room, whereas the volume of irradiated air refers only to the portion of the treatment space that is subject to irradiation. Occupiable space can have certain areas expected to be occupied. In various aspects, areas of an occupiable space that may be expected to be occupied can include seats, desks, tables, bar tops, waiting areas, patient beds, reception space, checkout areas, cubicles, stairways, walkways, and elevators. Typically, areas of expected human occupancy are less than 7 feet from a floor.

As used herein, the term “portable” refers to an apparatus that can be readily carried or transported from one location to another. For example, a portable apparatus can be a size and weight that allows it to be easily moved by a single person or a handcart. A portable apparatus can include handles, wheels, or other features that facilitate transport. A portable apparatus can be configured to be readily set up and broken down into easily transported components. In various examples, a portable apparatus can be separable into several components that can be assembled together by a single individual without specialized tools, training, or without destructive interaction with the space. A portable apparatus has advantages over installed fixtures and HVAC systems, for example, because one or more portable apparatus can be deployed as needed or adjusted based on the needs of the particular space being treated, variations in the use of the space (e.g., events), and the expected number of occupants and their location within the space. In various aspects, a portable device has the advantage of being user installable or serviceable without requiring specialized tradespeople. Another advantage, is that a suitably designed portable unit can be battery powered.

As used herein, the term “integrated” refers to the combination of two or more components into a single, unified apparatus. The integrated components can be physically integrated, such that they are physically connected together, or designed to fit together in a particular way that forms the integrated apparatus. The components can be integrated together by a number of means, for example, via mechanical fasteners, adhesives, welding, or molding. The integrated components can also be electrically integrated, such that they are electrically wired together or share the same power supply. The integrated components can also be functionally integrated in that the various components are configured to interoperate to achieve a particular end. In various aspects, an integrated apparatus can have the advantages of being compact, convenient, easy-to-use, and permits control of orientation of the integrated components. For example, integration of a far-UV source and an air stream unit can advantageously control the relative direction of an air stream and the relative position of the components with respect to emitted radiation. As another example, integration of a far-UV source and an air stream unit can be achieved via a shared mount, a shared stand, a shared base, or a shared housing.

As used herein, “non-pathogenic contaminants” can include, among other contaminants, one or more of ozone, oxygenated particles, oxygenated organic small molecules, oxygenated organic macromolecules and materials, oxygenated dust particles, non-pathogenic degradants of pathogens, and aggregated particles containing the same.

As used herein, the term “ultraviolet radiation” or “UV radiation” refers to electromagnetic radiation having one or more wavelength of about 100 nm to about 400 nm. UV radiation can further be subdivided into several categories based on wavelength: UVA (about 315 nm to about 400 nm), UVB (about 280 nm to about 315 nm), UVC (about 200 nm to about 280 nm), far-UV (about 200 nm to about 230 nm), vacuum UV (about 100 nm to about 200 nm), extreme UV (about 10 nm to about 100 nm). In various aspects and contexts, UV radiation can refer to various sub-ranges of UV radiation, for example 180 nm to 400 nm, 200 nm to 400 nm, 210 nm to 400 nm, 200 nm to 350 nm, 200 nm to 300 nm, or 210 nm to 280 nm. Radiation sources suitable for the presently described apparatus, system and method would not typically emit vacuum UV or extreme UV. As such, in various aspects the UV radiation source can be a UV radiation source that does not emit one or more type of UV light, such as a radiation source that does not emit vacuum UV or extreme UV. For example, the UV radiation source can emit radiation consisting of one or more wavelengths between 200 nm and 400 nm. In various aspects, UV radiation sources are configured such that they do not emit vacuum UV. A given radiation source can emit a spectrum having wavelengths according to multiple categories of light. For example, a far-UV radiation source may emit radiation having wavelengths corresponding to UVA, UVB, or UVC.

As used herein, “irradiance” is a measurement of power delivered to a surface per unit area, typically in units of ‘W/m²’ (watts per square meter), m W/m² or μW/cm². The surface can be an actual surface, e.g., of an object, or a given imaginary surface. Irradiance is one approach to describing the intensity of light. As a surface with a fixed area moves away from a light source the power delivered to it decreases as the square of the distance. Irradiance values depend on the orientation of the surface relative to the light source, declining with the difference between the surface's orientation and the orientation that would place it normal to incident rays from the light source. In some contexts, when irradiance is described based on a given distance from the radiation source, the surface can be understood to be an imaginary planar area at a distance from a radiation source and oriented facing the light source so as to be normal to incident rays from the source. In other contexts, when irradiance is described with respect to an object, the relevant surface can be understood to correspond to the surface orientation of such object, which can include walls, floors, ceilings, objects, or persons. Irradiance can be arrived at from a radiance value by specifying a distance and orientation for the irradiance value. Irradiance can also be arrived at from total optical power with knowledge of the distance from the source, orientation of the surface, and the relative position of the surface in the radiation pattern of the source. Under a fixed spectra this can be equated with a certain photon arrival rate for a certain surface in photons/(second·cm²). Irradiance as used herein is used in a ‘radiant’ context integrated over an entire relevant UV range; typically, for far-UV radiation it is sufficient to integrate across 180 nm to 400 nm. As also used herein, “total irradiance” refers to such irradiance across the 180-400 nm wavelength range, typically in units of W/cm² at a given distance from the light source. Occasionally, when specified, it can be useful to refer to irradiance of specific wavelengths or of a specific spectral series, such as a 222 nm or narrow band 222 nm light. In various aspects, irradiance, total irradiance, and the like can be useful for determining an instantaneous amount of radiation provided to a surface or for determining an average amount of radiation provided to a surface over a given period of time. Irradiance, total irradiance, and the like can also be used to measure or determine the disinfectant efficacy of a far-UV lamp, which can contain an optical filter, as well as its contribution to effective air changes per hour (eACH) in a given space, where such determinations can optionally be weighted based on the spectral profile of the light. In various aspects, irradiance, total irradiance, and the like can be useful for controlling disinfectant power of the far-UV lamp, such as via maintaining operation within a given germicidal efficacy profile and maintaining operation within a given safety profile.

As used herein, “total optical power”, “total optical flux” or “total optical power flux” refers to the photon generation rate of a UV radiation source across the 180-400 nm wavelength range in units of mW. It can be used to describe the disinfectant power of a lamp. This can be used as a measurement of the total power output of a UV radiation source or other device. This is distinct from the ‘wall power’ consumption of the lamp which is not related to this term in any meaningful way besides bounding it above.

As used herein, “fluence rate” is a measurement of power delivered to the surface area of a sphere, typically in units of “W/m²” (watts per square meter), mW/m² or μW/cm². This metric can be similar to “irradiance” and can share the same unit. However, whereas irradiance measures the rate of energy delivery to an oriented surface, fluence rate measures the rate of energy delivery to the surface of a sphere, and is therefore not dependent on the orientation of the measurement in space. When an irradiance measurement is taken for a surface pointing directly at a radiation source (i.e. such that the surface is normal to incident rays) and in the absence of reflections, the irradiance measurement and fluence rate measurements for that point will match. Another way to think of this is that irradiance only measures photons passing through a surface from one direction, and fluence rate measures photons passing through the surface of a spherical volume from any direction. Fluence rate as used herein is used in a ‘radiant’ context integrated over a relevant UV spectral range, e.g., often conveniently integrated across 180 nm to 400 nm. In various aspects, fluence rate is useful for determining an instantaneous amount of radiation provided to a volume or for determining an average amount of radiation provided to a volume over a given period of time. In various aspects, fluence rate is useful for determining instantaneous viral inactivation power of a UV light source, and correlates to eACH for the measured volume.

As used herein, “fluence” is a measurement of energy delivered to the surface area of a sphere in units of “J/m²” (joules per square meter.) Fluence measures the energy delivered to the surface of a sphere, and is therefore not dependent on the orientation of the measurement in space. Fluence is used herein in a ‘radiant’ context integrated over a relevant UV spectral range, e.g., often conveniently integrated across 180 nm to 400 nm. Fluence can be useful for correlating to an inactivation fraction for particles in a certain volume. In some aspects, it can be useful to determine fluence based on assuming a 222 nm narrow-band light source or based on a weighted integration of a multispectral source. Spectral weighting factors can be selected as suitable for a given use case: for example contribution to exposure limits, germicidal efficacy, or ozone generation. For example, when determining exposure in the context of a pathogen inactivation function, a narrow-band 222 nm source may be approximated as 222 nm light compared against an inactivation function for 222 nm light. A broader band spectrum of a UV light source may be integrated across the broad band spectrum and weighted at each wavelength according to how each wavelength contributes to exposure relative to 222 nm light.

As used herein, air changes per hour or “ACH” refers to the number of times the entire volume of air within a given space, e.g., a room, is replaced with filtered air in one hour. As such, ACH can be determined from CFM×60/volume of the space. In various contexts, ACH can be better understood by referring to ASHREA Standard 62 and ANSI Standard 136, each of which are incorporated by reference herewith.

As used herein, effective air changes per hour or “eACH” corresponds to the rate decontaminated air is provided to the air taking into account both air changes and inactivation of pathogens by germicidal irradiation. In the context of infectious disease, eACH can serve to equate the effect of inactivating pathogens and removing pathogens. The resulting value is an ACH equivalent determined by combining the ACH of the given space together with an irradiation-derived ACH equivalent value. In context of inactivating pathogens, the eACH refers to an ACH equivalent number of times the entire volume of air within a given space, e.g., a room, would need to be replaced with filtered air in one hour to obtain an equivalent result with respect to eliminating active pathogens.

As used herein, “effective irradiance” refers to the contribution of a given device to exposure limits, which can dictate the number of lamps suitable for deployment in a given space or dictate the allowable exposure time which a light may be used in an occupied space. As described herein, effective irradiance, “E_ff”, or “E_s”, can be calculated by measuring pointwise irradiance between 180-400 nm weighted by a factor corresponding to potential damage to the eyes. The weighting factor can be derived from the ACGIH exposure limit curve for eyes or the IES exposure limit curve for eyes.

### Far-UV Radiation Source

The apparatus of the present disclosure includes a far-UV radiation source, which is useful for inactivating airborne pathogens. The far-UV radiation source can utilize various technologies, including one or more bulbs, light-emitting diodes (LEDs), or other sources configured to emit UV radiation in an amount effective to inactivate pathogens in air.

The far-UV radiation source includes one or more excimer bulb. Excimer bulbs include a combination of a rare gas and halogen which emits one or more wavelength of far-UV light when excited by an electrical stimulus. Excimer bulbs excitation can be triggered in various ways including, for example, glow discharge, pulsed discharge, dielectric barrier discharge, short arc, or combinations thereof. In various examples, the far-UV radiation source comprises a KrCl excimer bulb or a KrBr excimer bulb. The far-UV radiation source generates and emits radiation comprising one or more wavelengths of about 210 nm to about 230 nm but may also emit other wavelengths as well. In various examples, the far-UV radiation source can emit UV wavelengths other than far-UV. In some examples, it can emit visible light. In further examples, the excimer bulb generates and emits 222 nm light. Various shapes of bulbs are possible. Bulbs are typically substantially tube-like, and can be rod-shaped, oblong, toroidal, or flattened versions of the same. The far-UV radiation source can include a bulb having an annular body having an outer surface and defining an internal discharge cavity, wherein the annular body has a major axial dimension and a minor radial dimension, and comprises an electrode in the internal discharge cavity that traverses along the major axial dimension of the annular body.

The far-UV radiation source can include a housing, the form of which is not particularly restrictive. Typically, the far-UV radiation source includes a housing containing one or more bulb. In various aspects, the housing partially encloses the one or more bulb and provides at least one opening through which radiation cam be emitted. For example, the housing can be a box where one face has an opening that is covered by a UV-transparent material serving as a protective window.

The far-UV radiation source can include any number of optical components. The window can include an optical filter, which can be useful for blocking potentially harmful wavelengths. The inside of the housing can be furnished with reflectors to direct light out the housing and maximize emitted radiation. The far-UV radiation source can include one or more component for adjusting the direction, intensity, or focus of the emitted light. For example, the far-UV radiation source can include a diffusor or lens.

Broadcast pattern refers to the shape of radiation emitted from the far-UV radiation source, which can be defined by a broadcast angle (spread) from each light source. The broadcast boundary corresponds to the edges of the broadcast pattern. Window material and any optical filter can restrict the broadcast angle due to reduced transmittance of off-axis light in many materials. In some aspects, the shape of the housing or optical components can limit the broadcast angle. For example, the broadcast angle can be altered by configuring housing and reflectors to control the angle of incidence on the window. Unless otherwise specified, the broadcast pattern and broadcast angle are determined based on substantial emission of 222 nm light. For irradiation airborne pathogens, a wide broadcast pattern can be advantageous for irradiation of a larger air space. In various contexts, broadcast pattern or radiation pattern of a UV light source can reflect the relative strength of total optical power per unit solid angle. A broadcast pattern measurement can also describe a general direction that the light source shines. Broadcast patterns can be described and measured as the irradiance values of the inside of a sphere with the light source placed at its center. A complete broadcast pattern measurement allows the calculation of irradiance and fluence rate for any point relative to the light source, not only those directly in front of it. See document ANSI/IES LM-75-19, a copy of which is incorporated by reference herewith.

The one or more far-UV radiation source can irradiate an occupiable space with a maximum irradiance at a value from about 0.5 μW/cm² to about 60 μW/cm² at a distance of 1, 2, 3, 4, 5, 6, 7, 8, 9, or 10 meters from the far-UV radiation source. For example, the maximum irradiance can be about or less than 0.5 μW/cm², 1.0 μW/cm², 1.5 μW/cm², 2.0 μW/cm², 2.5 μW/cm², 3.0 μW/cm², 3.5 μW/cm², 4.0 μW/cm², 4.5 μW/cm², 5.0 μW/cm², 5.5 μW/cm², 6.0 μW/cm², 6.5 μW/cm², 7.0 μW/cm², 7.5 μW/cm², 8.0 μW/cm², 8.5 μW/cm², 9.0 μW/cm², 9.5 μW/cm², 10 μW/cm², 11 μW/cm², 12 μW/cm², 13 μW/cm², 14 μW/cm², 15 μW/cm², 16 μW/cm², 17 μW/cm², 18 μW/cm², 19 μW/cm², 20 μW/cm², 21 μW/cm², 22 μW/cm², 23 μW/cm², 24 μW/cm², 25 μW/cm², 26 μW/cm², 27 μW/cm², 28 μW/cm², 29 μW/cm², 30 μW/cm², 31 μW/cm², 32 μW/cm², 33 μW/cm², 34 μW/cm², 35 μW/cm², 36 μW/cm², 37 μW/cm², 38 μW/cm², 39 μW/cm², 40 μW/cm², 41 μW/cm², 42 μW/cm², 43 μW/cm², 44 μW/cm², 45 W/cm², 46 μW/cm², 47 μW/cm², 48 μW/cm², 49 μW/cm², 50 W/cm², 51 μW/cm², 52 μW/cm², 53 μW/cm², 54 μW/cm², 55 μW/cm², 56 μW/cm², 57 μW/cm², 58 μW/cm², 59 μW/cm², or 60 μW/cm², or any range of values therein, at a distance of 1 meter from the far-UV radiation source or at a nearest distance between the far-UV radiation source and an location of an expected occupant. In some aspects, the one or more far-UV radiation source can irradiate an occupiable space with 10 μW/cm², 11 μW/cm², 12 μW/cm², 13 μW/cm², 14 μW/cm², or 15 W/cm² at a distance of 1 meter from the far-UV radiation source or at a nearest distance between the far-UV radiation source and a location of an expected occupant.

The one or more far-UV radiation source can irradiate an occupiable space with a fluence rate of about 0.5 μW/cm² to about 60 μW/cm² at a distance of 1, 2, 3, 4, 5, 6, 7, 8, 9, or 10 meters from the far-UV radiation source. For example, the fluence rate can be about or less than 0.5 μW/cm², 1.0 μW/cm², 1.5 μW/cm², 2.0 μW/cm², 2.5 μW/cm², 3.0 μW/cm², 3.5 μW/cm², 4.0 μW/cm², 4.5 μW/cm², 5.0 μW/cm², 5.5 μW/cm², 6.0 μW/cm², 6.5 μW/cm², 7.0 μW/cm², 7.5 μW/cm², 8.0 μW/cm², 8.5 μW/cm², 9.0 μW/cm², 9.5 μW/cm², 10 μW/cm², 11 μW/cm², 12 μW/cm², 13 μW/cm², 14 μW/cm², 15 μW/cm², 16 μW/cm², 17 μW/cm², 18 μW/cm², 19 μW/cm², 20 μW/cm², 21 W/cm², 22 μW/cm², 23 μW/cm², 24 μW/cm², 25 μW/cm², 26 μW/cm², 27 W/cm², 28 μW/cm², 29 W/cm², 30 μW/cm², 31 μW/cm², 32 μW/cm², 33 μW/cm², 34 μW/cm², 35 μW/cm², 36 μW/cm², 37 μW/cm², 38 μW/cm², 39 μW/cm², 40 μW/cm², 41 μW/cm², 42 μW/cm², 43 μW/cm², 44 μW/cm², 45 μW/cm², 46 μW/cm², 47 μW/cm², 48 μW/cm², 49 μW/cm², 50 μW/cm², 51 μW/cm², 52 μW/cm², 53 μW/cm², 54 μW/cm², 55 μW/cm², 56 μW/cm², 57 μW/cm², 58 μW/cm², 59 μW/cm², or 60 μW/cm², or any range of values therein, at a distance of 1 meter from the far-UV radiation source or at a nearest distance between the far-UV radiation source and an location of an expected occupant. In some aspects, the one or more far-UV radiation source can irradiate an occupiable space with a fluence rate of 10 μW/cm², 11 μW/cm², 12 μW/cm², 13 μW/cm², 14 μW/cm², or 15 μW/cm² at a distance of 1 meter from the far-UV radiation source or at a nearest distance between the far-UV radiation source and a location of an expected occupant.

The one or more far-UV radiation source can irradiate an occupiable space with a total optical power of about or less than 4 mW, 5 mW, 6 mW, 7 mW, 8 mW, 9 mW, 10 mW, 15 mW, 20 mW, 25 mW, 30 mW, 35 mW, 40 mW, 45 mW, 50 mW, 55 mW, 60 mW, 65 mW, 70 mW, 75 mW, 80 mW, 85 mW, 90 mW, 95 mW, 100 mW, 105 mW, 110 mW, 115 mW, 120 mW, 125 mW, 130 mW, 135 mW, 140 mW, 145 mW, 150 mW, 155 mW, 160 mW, 165 mW, 170 mW, 175 mW, 180 mW, 185 mW, 190 mW, 195 mW, 200 mW, 205 mW, 210 mW, 215 mW, 220 mW, 225 mW, 230 mW, 235 mW, 240 mW, 245 mW, 250 mW, 255 mW, 260 mW, 265 mW, 270 mW, 275 mW, 280 mW, 285 mW, 290 mW, 295 mW, 300 mW, 305 mW, 310 mW, 315 mW, 320 mW, 325 mW, 330 mW, 335 mW, 340 mW, 345 mW, 350 mW, 355 mW, 360 mW 15 mW, 20 mW, 25 mW, 30 mW, 35 mW, 40 mW, 45 mW, 50 mW, 55 mW, 60 mW, 65 mW, 70 mW, 75 mW, 80 mW, 85 mW, 90 mW, 95 mW, 100 mW, 101 mW, 102 mW, 103 mW, 104 mW, 105 mW, 106 mW, 107 mW, 108 mW, 109 mW, 110 mW, 111 mW, 112 mW, 113 mW, 114 mW, 115 mW, 116 mW, 117 mW, 118 mW, 119 mW, 120 mW, 121 mW, 122 mW, 123 mW, 124 mW, 125 mW, 126 mW, 127 mW, 128 mW, 129 mW, 130 mW, 131 mW, 132 mW, 133 mW, 134 mW, 135 mW, 136 mW, 137 mW, 138 mW, 139 mW, 140 mW, 145 mW, 150 mW, 155 mW, 160 mW, 165 mW, 170 mW, 175 mW, 180 mW, 185 mW, 190 mW, 195 mW, 200 mW, 205 mW, 210 mW, 215 mW, 220 mW, 225 mW, 230 mW, 235 mW, 240 mW, 245 mW, 250 mW, 255 mW, 260 mW, 265 mW, 270 mW, 275 mW, 280 mW, 285 mW, 290 mW, 295 mW, 300 mW, 305 mW, 310 mW, 315 mW, 320 mW, 325 mW, 330 mW, 335 mW, 340 mW, 345 mW, 350 mW, 355 mW, or 360 mW, or any range of values therein. For example, the one or more far-UV radiation source can irradiate an occupiable space with a total optical power of 120 mW.

The far-UV radiation source can have an optical filter. In various examples, the optical filter can substantially prevent transmittance of one or more wavelength outside of the range between about 200 nm to about 230 nm. The far-UV radiation source comprises an optical filter that substantially transmits one or more wavelength outside of the range between about 200 nm to about 230 nm. In more specific embodiments, the far-UV radiation source comprises an optical filter that substantially prevents transmittance of one or more wavelength outside of the range between about 200 nm to about 230 nm, but also substantially transmits one or more wavelength outside of the range between about 200 nm to about 230 nm.

Far-UV radiation sources are commercially available: for example, B1, B1.5, B2, C1, an C2 far-UV KrCl emitters from Ushio Inc. (Tokyo, Japan), a micro plasma KrCl emitter from Eden Park Illumination (Champaign, IL), a Lumenizer KrCl emitter by Lumen Labs (Round Rock, Tx), or a EL-09 KrCl UVC emitter module from Excelitas (Waltham, MA).

### Air Stream Unit

The apparatus of the present disclosure can include an air stream unit. The air stream unit includes one or more air intake, one or more air exhaust, one or more air flow chamber, and one or more fan. The air stream unit can also include one or more mechanical filter, one or more scrubber, or both. The air stream housing provides both a protective and functional purpose. For example, it can protect internal components and can also be designed to control air flow. It can be constructed from plastic, metal, resin, or composite materials. In various aspects, the housing is rigid. The air stream housing can have one or more air intake and one or more air exhaust. The air intake and air exhaust are openings in the air stream housing that permit air to flow between external space and the space inside the air stream unit.

The air stream unit can be integrated with the far-UV radiation source via a fastener, an adhesive, an interlocking part, welding, or being molded together with an exterior surface of the air stream housing. The air stream unit can be integrated with the far-UV radiation source via a shared stand or mount. The air stream unit can be integrated with the far-UV radiation source via a shared power supply. The air stream housing can be positioned to approximate an edge of the broadcast pattern of the far-UV radiation source. In some examples, a dimension of the air stream housing, air intake, or air exhaust is approximately equal to a dimension of the far-UV radiation source or a bulb of the far-UV radiation source. In various examples, the air intake or air exhaust that is adjacent to the far-UV radiation source is substantially rectangular having an edge substantially parallel to an edge of the far-UV radiation source or a reflector, bulb, or lens of the far-UV radiation source. In some examples, the air intake or air exhaust that is adjacent to the far-UV radiation source is annular and circumscribes the far-UV radiation source. In yet further examples, the air stream housing and the far-UV radiation source when integrated provides provide a cuboid or cylindrical shape. In various further examples, the far-UV radiation source is located within 24 inches of the air intake or air exhaust. The apparatus can be configured for attaching the air stream housing to a return or a supply of an HVAC system

The air stream unit is configured to displace irradiated air proximate to the far-UV radiation source. The air intake can be adjacent to the far-UV radiation source and the air stream housing can be configured to capture irradiated air proximate to the far-UV radiation source. In some aspects, the air exhaust can be directed away from the far-UV radiation source and can optionally be directed to a ceiling, wall, floor, or deflector to circulate air in an occupiable space. The air intake can also be adjacent to the far-UV radiation source and the air stream housing can be configured to exhaust the air stream away from irradiated air proximate to the far-UV radiation source. The air exhaust can be adjacent to the far-UV radiation source and the air stream housing can be configured to exhaust the air stream into a space irradiated by the far-UV radiation source. In further aspects, the air stream housing can be configured to displace irradiated air in a direction substantially parallel to a direction of the emitted radiation. In yet further aspects, the air stream housing is configured to capture irradiated air and displace it in a direction substantially opposite to a direction of the emitted radiation. In another aspect, the air stream housing is configured to displace irradiated air in a direction substantially perpendicular to a direction of the emitted radiation. In various examples, the air intake and air exhaust provide one ore more rotary vortices in a room where the apparatus is positioned.

In further examples, the air stream unit provides an air stream at the air intake and/or the air exhaust of 60 ft³/min to 5,000 ft³/min. In various examples, the air stream at air intake and/or the air exhaust has a volumetric flow rate of at least or about 60 ft³/min, 70 ft³/min, 80 ft³/min, 90 ft³/min, 100 ft³/min, 125 ft³/min, 150 ft³/min, 175 ft³/min, 200 ft³/min, 250 ft³/min, 300 ft³/min, 350 ft³/min, 400 ft³/min, 450 ft³/min, 500 ft³/min, 550 ft³/min, 600 ft³/min, 650 ft³/min, 700 ft³/min, 800 ft³/min, 900 ft³/min, 1,000 ft³/min, 2,000 ft³/min, 3,000 ft³/min, 4,000 ft³/min, or 5,000 ft³/min. In some aspects, the air stream housing is configured to provide a diffuse exhaust. In further aspects, the air stream unit is configured such that the air exhaust will not provide an airflow wall, a partition airflow in an occupied space, or other means for minimizing air mixing, circulation, and movement in a treatment space. In various aspects, the air stream housing is configured for facilitating the movement or scattering of droplets, aerosols, pathogens, and particles in air.

In various examples, the far-UV apparatus can provide a clean air delivery rate (CADR) of about, or at least, 50 m³/hr, 100 m³/hr, 150 m³/hr, 200 m³/hr, 250 m³/hr, 300 m³/hr, 350 m³/hr, 400 m³/hr, 450 m³/hr, 500 m³/hr, 550 m³/hr, 600 m³/hr, 650 m³/hr, 700 m³/hr, 750 m³/br, 800 m³/hr, 850 m³/hr, 900 m³/hr, 950 m³/hr, or 1,000 m³/hr. In various examples, the far-UV apparatus can provide a volumetric air flow of about 50 m³/hr, 100 m³/hr, 150 m³/hr, 200 m³/hr, 250 m³/hr, 300 m³/hr, 350 m³/hr, 400 m³/hr, 450 m³/hr, 500 m³/hr, 550 m³/hr, 600 m³/hr, 650 m³/hr, 700 m³/hr, 750 m³/hr, 800 m³/hr, 850 m³/hr, 900 m³/hr, 950 m³/hr, 1,000 m³/hr, 1,100, 1,200 m³/hr, 1,300 m³/br, 1,400 m³/hr, 1,500 m³/hr, 1,600 m³/hr, 1,650 m³/hr, 1,700 m³/hr, 1750 m³/hr, 1,800 m³/hr, 1,850 m³/hr, 1,900 m³/hr, 1950 m³/hr, or 2,000 m³/hr.

An air stream unit can provide air changes per hour (ACH) in a given space of 1-30 ACH. In further aspects, the apparatus as a whole can provide air changes per hour (ACH) in a given space of 1-30 ACH.

The far-UV radiation source can provide effective air changes per hour (eACH) in a given space of at least, or about, 4 eACH to 2,000 eACH. For example, the far-UV radiation source can provide 90 eACH. In further aspects, the apparatus as a whole, adjusting for the effect of its combined far-UV radiation source and air stream unit, can provide an effective air changes per hour (eACH) in a given space of 5 eACH to 3,5000 eACH. For example, the apparatus can provide about 100 eACH. In some examples, the far-UV apparatus can provide greater eACH than would be achieved by use of a separate far-UV radiation source and air purifier that are not integrated together as presently described. In some examples, the far-UV apparatus can provide greater eACH than would be achieved by use of a separate far-UV radiation source and a stand-alone air purifier having a HEPA filter.

The apparatus, system, and methods described herein can be configured to provide an air stream through the irradiated air space in various orientations relative to the predominant direction of UV irradiation, including a pull orientation (i.e., sucking air towards the far-UV radiation source), a push orientation (i.e., blowing air away from the far-UV radiation source), perpendicular orientations (i.e., sucking or blowing air perpendicular to the direction of radiation from the far-UV radiation source), a push-pull orientation (e.g., where intake and exhaust are both facing in the direction that UV radiation is transmitted so as to provide a circulating or vortex movement of air), orientations where air is exhaust away from the direction of intake including toward a wall or other boundary surface to provide circular air flow in a room, orientations where air is exhausted toward the area of irradiation, orientations where air is pulled into an intake from an area with a wall or other boundary surface to provide circular air flow in a room, and other orientations. Pulling configurations can pull contaminants from lightly irradiated or non-irradiated regions to highly irradiated regions, it can provide movement that disrupt certain conditions that lead to secondary product formation, it can reduce the amount of time that certain vulnerable particles are subjected to UV radiation, it can direct secondary products to a filter or a scrubber, or any combination thereof. Pushing configurations can provide clean air that disrupts certain conditions that lead to secondary product formation, it can provide movement that disrupt certain conditions that lead to secondary product formation, it can reduce the amount of time that certain vulnerable particles are subjected to UV radiation, and it can provide room air currents that move contaminants out of stagnant regions. Purified air can be exhausted in a direction that provides circulation of air through the space, moves contaminants out of stagnant regions or non-irradiated regions, and exhausted air can be processed or measured such that it represents a known value that can be directed to a desired location. For example, in various aspects, air is exhausted out the back of a far-UV apparatus to move air that is not subjected to irradiation and facilitate its circulation through the room. As another example: in various aspects, purified air is exhausted toward the area of irradiated air to break up localized concentrations of particles that are undesirable or susceptible to UV-induced formation of harmful byproducts. A sufficiently robust air stream can result in a recirculating loop in which purified air is exhausted and recirculated through the apparatus without fully mixing throughout the space. For example, an apparatus configured to exhaust purified air in the direction of irradiation can break up localized concentrations of pathogens, VOCs, ozone, precursors of secondary contaminants, and secondary contaminants, and efficiently direct them to an intake even when the intake is located on the rear or side of the apparatus or when the apparatus is configured for placement near a wall.

### Filter System

The apparatus of the present disclosure can include a filter system. As used herein, a “filter system” or “purification system” refers to the one or more mechanical filters and one or more scrubbers, which ever components are present. A filter system can comprise a plurality of mechanical filters, a plurality of scrubbers, one filter and one scrubber, a plurality of mechanical filters and one scrubber, or one scrubber and a plurality of mechanical filters. The components of the filter system are disposed within the air stream housing, either in or around the air intake, air exhaust, or air flow chamber.

The apparatus of the present disclosure can include a mechanical filter. A mechanical filter is designed to physically capture and remove particles from the air. The mechanical filter can be made of any variety of materials, such as fiberglass, natural fibers such as cotton, synthetic fibers such as polyester, borosilicate glass microfibers, electrostatically charged polypropylene fibers, spun polymer nanofibers, or metal mesh. In some examples, the mechanical filter is other than borosilicate microfibers.

The mechanical filter can have a particular particle size rating, such as a MERV rating or HEPA rating. MERV and HEPA ratings indicate the size of particles that can be a captured with a certain efficiency. MERV refers to minimum efficiency reporting value, which is typically based on a filter's ability to capture particles between 0.3 microns and 10 microns. Unless otherwise specified, MERV values correspond to ANSI/ASHRAE Standard 52.2-2017. In some contexts, the MERV value can be described with respect to 0.3-micron particles, specifically. In various examples, the filter can be a MERV 6, MERV 7, MERV 8, MERV 9, MERV 10, MERV 11, MERV 12, MERV 13, MERV 14, MERV 15, or MERV 16 filter. In further examples, the filter can have a MERV rating of less than 7, 8, 9, 10, 11, 12, 13, 14, 15, 16, or 17. HEPA filter refers to high efficiency particulate air filter, and include H10, H11, H12, H13, and H14 grades. Unless otherwise specified, HEPA designation and values corresponds to ISO 294363-1 2017. In some context, the HEPA designation or values can be described with respect to 0.3-micron particles, specifically. In some aspects, the filter can be a HEPA filter. In other aspects, the filter is other than a HEPA. In various examples, the apparatus does not include any HEPA filter. In yet further examples, the apparatus does not include any filter rated at MERV14 or greater per MERV Standard 52.2.

In various examples, the filter system consists of two MERV13 filters and optionally one or more scrubber.

Indeed, in various examples, the apparatus has the advantage of effectively utilizing lower cost filters or higher penetrating filters.

The one or more mechanical filter, or filter system as a whole, can have a penetration of 5% or greater at 0.3 microns. Typically, penetration values can be determined from 1-filter efficiency, where filter efficiency is based on the most penetrating particle size, or 0.3 microns. Penetration values can correspond to ISO 29463-1 2017. In further examples, the one or more mechanical filter, or filter system as a whole, can have a penetration of 5%, 6%, 7%, 8%, 9%, 10%, 11%, 12%, 13%, 14%, 15%, 16%, 17%, 18%, 19%, 20%, 25%, 30%, 35%, or 40% or greater at 0.3 microns. In yet further examples, one or more mechanical filter, or filter system as a whole, can have a penetration of up to 30%, 40%, 50%, or 60%. In various contexts, it can be useful to refer to E1 filtration per the ASHRAE 52.2 test, which refers to particles 0.3 microns to 1.0 microns, but can approximate penetration at 0.3 microns. An additional advantage of higher penetration filter media is lower pressure drop is involved for target results so less motor power needed and lower noise can be achieved.

Conventionally, lower penetration is considered to represent a better filter. However, in various aspects of the present invention, a mechanical filter, or filter system, having higher penetration can be advantageous. Surprisingly, the apparatus, systems, and methods described herein can benefit from a filter system, filters, and scrubbers having higher, rather than lower, penetration due to decoupling of the removal of pathogen contaminants from the removal of non-pathogenic contaminants. The area of germicidal UV treatment is in the space outside of the apparatus while the area of filter and/or scrubber is within an air stream unit within the apparatus. A air stream of fast moving air can be generated that is localized to the apparatus and proximate air of higher UV intensity. Higher penetration permits increased airflow near the radiation source and through the housing. It can also facilitate air circulation for multipass treatment.

The one or more mechanical filter, or filter system as a whole, can have a single pass efficiency of about 40% to about 95%. For example, the single pass efficiency can be about 40%, 45%, 50%, 55%, 60%, 65%, 70%, 75%, 80%, 85%, 90%, or 95%, or any range of values therein. In various examples, the single pass efficiency is 70%. In further examples, the one or more mechanical filter, or filter system as a whole, can have a single pass efficiency of about, or less than, 40%, 45%, 50%, 55%, 60%, 65%, 70%, 75%, 80%, 85%, 90%, or 95% and a pressure drop curve of 0.4 mm H₂O at 100 CFR to 2.0 mm H₂O at 500 CFM.

The one or more mechanical filter can have a filter media having a surface area of about 4 m² to 210 m². For example, the surface area can be about 4 m², 5 m², 6 m², 7 m², 8 m², 9 m², 10 m², 11 m², 12 m², 13 m², 14 m², 15 m², 16 m², 17 m², 18 m², 19 m², 20 m², 21 m², 22 m², 23 m², 24 m², 25 m², 26 m², 27 m², 28 m², 29 m², 30 m², 31 m², 32 m², 33 m², 34 m², 35 m², 36 m², 37 m², 38 m², 39 m², 40 m², 41 m², 42 m², 43 m², 44 m², 45 m², 46 m², 47 m², 48 m², 49 m², 50 m², 51 m², 52 m², 53 m², 54 m², 55 m², 56 m², 57 m², 58 m², 59 m², 60 m², 61 m², 62 m², 63 m², 64 m², 65 m², 66 m², 67 m², 68 m², 69 m², 70 m², 71 m², 72 m², 73 m², 74 m², 75 m², 76 m², 77 m², 78 m², 79 m², 80 m², 90 m², 100 m², 110 m², 120 m², 130 m², 140 m², 150 m², 160 m², 170 m², 180 m², 190 m², 200 m², or 210 m². For example, the one or more mechanical filters can together have filter media having a surface area of 42 m². In various aspects, the mechanical filter utilizes pleating to fit greater filter media surface area in a smaller filter cartridge. In some aspects, the filter is pleated so as to fit within a filter cartridge that is 0.03 m² to 8 m², which corresponds the surface area of the cartridge face. In some aspects, the filter cartridge is 0.2 m². In various aspects, the filter media can have thickness of 0.05 mm to 2.5 mm, for example 0.3 mm. The filter cartridge can have a depth of 7 mm to 125 mm, for example 25 mm. In a yet further example, the filter cartridge can be 0.2 m×0.2 m×0.025 m.

The one or more mechanical filter can have a face velocity of 0.05 m/sec to 15 m/sec. That is, the one or more mechanical filters can be subject to said face velocity, but also rated, configured, or selected for receiving such face velocity. For example, a mechanical filter can have a face velocity of 0.05 m/sec, 0.10 m/sec, 0.15 m/sec, 0.20 m/sec, 0.25 m/sec, 0.30 m/sec, 0.35 m/sec, 0.40 m/sec, 0.45 m/sec, 0.50 m/sec, 0.55 m/sec, 0.60 m/sec, 0.65 m/sec, 0.70 m/sec, 0.75 m/sec, 0.80 m/sec, 0.85 m/sec, 0.90 m/sec, 0.95 m/sec, 1 m/sec, 2 m/sec, 3 m/sec, 4 m/sec, 5 m/sec, 6 m/sec, 7 m/sec, 8 m/sec, 9 m/sec, 10 m/sec, 11 m/sec, 12 m/sec, 13 m/sec, 14 m/sec, or 15 m/sec.

In various aspects, the one or more mechanical filter can be rated as having a maximum flow rate of about or at least 1000 ft³/min, 2000 ft³/min, 3000 ft³/min, 4000 ft³/min, or 5000 ft³/min. The one or more mechanical filter can be rated as having a pressure drop of 0.25 mm H₂O to 25 mm H₂O. For example, the pressure drop can be 0.25 mm H₂O, 0.30 mm H₂O, 0.35 mm H₂O, 0.40 mm H₂O, 0.45 mm H₂O, 0.50 mm H₂O, 0.55 mm H₂O, 0.60 mm H₂O, 0.65 mm H₂O, 0.70 mm H₂O, 0.75 mm H₂O, 0.80 mm H₂O, 0.85 mm H₂O, 0.90 mm H₂O, 0.95 mm H₂O, 1 mm H₂0, 2 mm H₂O, 2 mm H₂0, 4 mm H₂O, 5 mm H₂0, 6 mm H₂O, 7 mm H₂0, 8 mm H₂O, 9 mm H₂O, 10 mm H₂0, 11 mm H₂O, 12 mm H₂O, 13 mm H₂O, 14 mm H₂O, 15 mm H₂O, 16 mm H₂0, 17 mm H₂0, 18 mm H₂O, 19 mm H₂0, 20 mm H₂0, 21 mm H₂0, 22 mm H₂0, 23 mm H₂O, 24 mm H₂O, or 25 mm H₂O, or a range of values therein. In various aspects, the one or more mechanical filter can have a pressure drop of 1.00 mm H₂O or less. The pressure drop can be 0.25 mm H₂O, 0.30 mm H₂O, 0.35 mm H₂O, 0.40 mm H₂O, 0.45 mm H₂O, 0.50 mm H₂O, 0.55 mm H₂O, 0.60 mm H₂0, 0.65 mm H₂O, 0.70 mm H₂O, 0.75 mm H₂O, 0.80 mm H₂O, 0.85 mm H₂0, 0.90 mm H₂O, 0.95 mm H₂O, 1 mm H₂0, 2 mm H₂0, 2 mm H₂0, 4 mm H₂O, 5 mm H₂O, 6 mm H₂O, 7 mm H₂O, 8 mm H₂O, 9 mm H₂O, 10 mm H₂O, 11 mm H₂O, 12 mm H₂O, 13 mm H₂O, 14 mm H₂O, 15 mm H₂O, 16 mm H₂O, 17 mm H₂O, 18 mm H₂O, 19 mm H₂O, 20 mm H₂O, 21 mm H₂O, 22 mm H₂O, 23 mm H₂O, 24 mm H₂O, or 25 mm H₂O, or a range of values therein, at a flow rate of at least 50, 100, 150, 200, 250, 300, 350, 400, 450, 500, 550, 600, 650, 700, 750, 800, 850, 900, 950, or 100 CFM. The one or more mechanical filter can have a flow rate of at least 50 CFM at a pressure drop of 0.25 mm H₂O or less.

The apparatus of the present disclosure can include a scrubber. A scrubber is designed to remove contaminants from air using a chemical process or physicochemical processes, such as adsorption. The scrubber can made of a variety of materials including, but not limited to, activated carbon, manganese dioxide, graphene, carbon nanoparticles, homogenous metal doped catalyst, heterogenous doped metal, or zeolite or other absorbent microporous materials. Catalytic metals can include platinum, palladium, rhodium, and manganese. The scrubber can be a passive scrubber or an active scrubber. For example, the scrubber can be an active scrubber that is powered by a power supply shared with the far-UV radiation source and the fan system. The scrubber can be configured to remove ozone from air. The scrubber can be configured to remove ozone derivatives or products arising from reaction with ozone. The scrubber can be configured to remove highly oxygenated organic molecules (HOMs) from air. The scrubber can be configured to particles resulting from clustered highly oxygenated organics. The scrubber can be located in the air stream unit, such as at an air intake, air exhaust, or air flow chamber.

Scrubbers and scrubber materials are commercially available: for example, granular activated carbon available such as HS-AC, granular zeolite HS-600, and mixtures of granular activated carbon and zeolite such as XB-17, are each available from Hydrosil International Ltd (East Dundee, IL). Other available materials include ozone decomposition catalyst on aluminum honeycomb such as XT-CAT-07 available from Hunan Xintan New Material Co., Ltd. In various aspects, one or more scrubbers may have a pressure drop of about or less than 0.25 to 10 inches H₂O/per foot for a superficial velocity of 25 to 100 ft/min. In various aspects, one or more scrubbers may have a pressure drop corresponding to one or more of the filters described herein, or the scrubbers and filters taken together may have a pressure drop corresponding to one or more of the filters described herein. In various aspects, the scrubber or scrubber material is a manganese dioxide.

The present filter system can be configured particularly for removing non-pathogenic contaminants in air, including ozone, oxygenated particles, oxygenated organic small molecules, oxygenated organic macromolecules and materials, oxygenated dust particles, non-pathogenic degradants of pathogens, and aggregated particles containing the same.

The present filter system can be configured for multiple passes. In some examples, the one or more mechanical filter, or filter system as a whole, is not suitable as a single pass filter for achieving an ISO 20E or better using ISO 29463-1 2017.

### Fan System

The apparatus of the present disclosure can include a fan system. The fan system generates the air stream and moves air through the air stream unit. The fan system includes one or more fans. The fan can be a tubeaxial fan or a rotary impeller.

The one or more fans can be various sizes. In various aspects, the fan has a diameter that is about 30 mm to about 600 mm, including the fan housing. For example, the fan can have a diameter of about 40 mm, 60 mm, 80 nm, 92 mm, 120 mm, 140 mm, or 200 mm. In another aspect, the fan can be about 500-600 mm, including the fan housing. The fan can have a depth of about 10 mm to about 90 mm. The fan can be configured for about 500 rpm to about 5000 rpm. The one or more fans can have a noise profile of about 6 dB to about 30 dB.

The one or more fans can be configured for an air flow of about 8 m³/h to about 500 m³/h. For example, the fan can be configured for an air flow of about or at least 10 m³/h, 20 m³/h, 30 m³/h, 40 m³/h, 50 m³/h, 60 m³/h, 70 m³/h, 80 m³/h, 90 m³/h, 100 m³/h, 110 m³/h, 120 m³/b, 130 m³/h, 140 m³/h, 150 m³/h, 160 m³/h, 170 m³/h, 180 m³/h, 190 m³/h, 200 m³/h, 210 m³/h, 220 m³/h, 230 m³/h, 240 m³/h, 250 m³/h, 260 m³/h, 270 m³/h, 280 m³/h, 290 m³/h, 300 m³/h, 310 m³/h, 320 m³/h, 330 m³/h, 340 m³/h, 350 m³/h, 360 m³/h, 370 m³/h, 380 m³/h, 390 m³/h, or 400 m³/h, or any range of integers therein. The fan system can together provide an air flow at an air inlet, an air exhaust, or both, of at least or about 50 m³/h, 60 m³/h, 70 m³/h, 80 m³/h, 90 m³/h, 100 m³/h, 110 m³/h, 120 m³/h, 130 m³/h, 140 m³/h, 150 m³/h, 160 m³/h, 170 m³/h, 180 m³/h, 190 m³/h, 200 m³/h, 210 m³/h, 220 m³/h, 230 m³/h, 240 m³/h, 250 m³/h, 260 m³/h, 270 m³/h, 280 m³/h, 290 m³/h, 300 m³/h, 310 m³/h, 320 m³/h, 330 m³/h, 340 m³/h, 350 m³/h, 360 m³/h, 370 m³/h, 380 m³/h, 390 m³/h, or 400 m³/h.

The one or more fans can be configured for a static pressure of about 0.40 mm H₂O to about 8.00 mm H₂O. For example, the fan can be configured for a static pressure of about 0.40 mm H₂O, 0.50 mm H₂O, 0.60 mm H₂O, 0.70 mm H₂O, 0.80 mm H₂O, 0.90 mm H₂O, 1.00 mm H₂O, 1.10 mm H₂O, 1.20 mm H₂O, 1.30 mm H₂O, 1.40 mm H₂O, 1.50 mm H₂O, 1.60 mm H₂O, 1.70 mm H₂O, 1.80 mm H₂O, 1.90 mm H₂O, 2.00 mm H₂O, 2.10 mm H₂O, 2.20 mm H₂O, 2.30 mm H₂O, 2.40 mm H₂O, 2.50 mm H₂O, 3.00 mm H₂O, 3.50 mm H₂O, 4.00 mm H₂O, 4.50 mm H₂O, 5.00 mm H₂O, 5.50 mm H₂O, 6.00 mm H₂O, 6.50 mm H₂O, or 7.00 mm H₂O, or any range of values therein.

The fan system can be configured for a total fan power of about, or less than, 5 W, 6 W, 7 W, 8 W, 9 W, 10 W, 11 W, 12 W, 13 W, 14 W, 15 W, 20 W, 25 W, 30 W, 35 W, 40 W, 45 W, 50 W, 55 W, 60 W, 65 W, 70 W, 75 W, 80 W, 85 W, 90 W, 95 W, 100 W, 110 W, 120 W, 130 W, 140 W, 150 W, 160 W, 170 W, 180 W, 190 W, or 200 W. One or more fans can be configured at about 5V to about 24V. In various aspects, one or more fans can be configured to draw about 0.2 W to about 10 W. For example, one or more fans can draw 0.6 W to about 6 W.

The fan system can include one or more brushless DC motor, a brushed DC motor, or an AC motor.

### Power Supply

The apparatus of the present disclosure can include a power supply. The power supply can be any device or system that provides electrical energy to the components of the apparatus, such as the far-UV radiation source, and fan system. The power supply can also provide power to any active filter or active scrubber. The power supply is typically internally located, but it can also be externally located. The power supply can include an AC/DC adapter. The power supply can also include circuitry for regulating voltage, current, power, timing of operation, or duration of operation in order to ensure safe operation. In some aspects, the power supply can include a battery.

In various aspects, the apparatus can include additional electrical components for data collection, system maintenance, and reporting. For example, the apparatus can have a sensor that collects spectrophotometric data, irradiance data, or spectral data of one or more far-UV radiation sources, air quality data, gas content data, sound data, light data, usage data, system status, maintenance-related data, room context data, room occupancy data, or any combination thereof. In another example, the apparatus can have a control system that receives sensor data and adjusts a performance parameter of the apparatus. In yet further examples, the apparatus can have a reporting system that collects sensor data and notifies a user of suggested maintenance or changes to a performance parameter.

In various aspects, the apparatus can include a sensor for detecting ozone. The ozone sensor can be located near an intake, an exhaust, a far-UV radiation source, or within an air chamber. In various aspects, the ozone sensor can be located within 1, 2, 3, 4, 5, 6, 7, 8, 9, 10, 11, or 12 inches from the housing. In various aspects, the ozone sensor can be located within 1, 2, 3, 4, 5, 6, 7, 8, 9, 10, 11, or 12 inches from an intake of the apparatus. In various aspects, the ozone sensor can be located within 1, 2, 3, 4, 5, 6, 7, 8, 9, 10, 11, or 12 inches from an exhaust of the apparatus. In various aspects, the ozone sensor can be located within 1, 2, 3, 4, 5, 6, 7, 8, 9, 10, 11, or 12 inches from the far-UV radiation source. For example, the ozone sensor can be located within 2 inches from an exhaust of the apparatus.

In some aspects, the air stream can be allowed to pass over the power supply or over a heat sink for the power supply in order to facilitate cooling. Yet, in other aspects, the power supply is separate from the air stream.

### Integration

The presently described apparatus involves a far-UV radiation source integrated with an air stream unit. The far-UV radiation source can be physically integrated with the air stream unit, for example via a fastener, an adhesive, an interlocking part, welding, or by being molded together. As another example, the air stream housing and the housing of the far-UV radiation source are attached, have a shared wall, or have a shared edge. The far-UV radiation source can be located near an air intake or air exhaust of the air stream housing. As yet another example, the far-UV radiation source and the air stream unit can be physically integrated by sharing a support, such as a stand or mount. The far-UV radiation source and air stream unit can be configured such that the air stream unit provides an air stream oriented relative to the pattern of radiation broadcasted by the far-UV radiation. The far-UV radiation source can also be integrated with the air stream housing by sharing a power supply.

The far-UV radiation source can be functionally integrated with the air stream housing by configuration of the various components to interoperate. In various aspects, the far-UV radiation source and air stream unit are configured to interoperate in a manner such that either component separately would not suffice to inactive pathogens in air while avoiding dangerous levels of ozone or ozone-derived contaminants.

### Deployment

The apparatus of the present disclosure can have a support, which can be fixed or adjustable. A fixed or adjustable support can be useful for positioning the device at a height and angle to more effectively irradiate the treatment space and also to move air near areas of high pathogen transmission, particularly the air about four to seven feet off the ground closest to occupants. The stand can also permit placement of the apparatus near the ceiling, near where the walls meet the ceiling, or near the upper corners of a room, and so can advantageously be positioned to deflect air to increase air circulation through the room. The support can be a stand, which can be configured for use on a floor, tabletop, or countertop. An adjustable support can be an extensible pole or arm to which the far-UV apparatus can be mounted. An adjustable support can also include legs that are collapsible, adjustable, telescoping, or folding. For example, the adjustable support can be collapsible or foldable to permit easy transport. The adjustable support can also include one or more mechanism for locking it into a particular position. The adjustable support can also have a weighted base.

One or more apparatus can be deployed to inactivate pathogens in air by irradiating a treatment space with far-UV light. Inactivation of pathogens is related to both the intensity of radiation and the amount of time the pathogen is irradiated. A user can utilize multiple apparatus to provide a predetermined intensity of radiation based on the size of the treatment space.

The far-UV apparatus can utilize the support to provide about 0.5 μW/cm² to about 60 μW/cm² at one or more occupant or area of expected occupancy, for example by permitting the far-UV radiation source to be placed away from occupants. For example, the support can facilitate raising the far-UV radiation source or attaching it to the ceiling or walls of a space.

The far-UV apparatus is configured such that when deployed in an occupied space, the emitted radiation lacks the intensity, power, irradiance, fluence rate, fluence, effective irradiance, or total irradiance necessary for inactivating pathogens on the skin or other surface of an occupant.

The far-UV apparatus is configured such that when deployed in an occupied space, the emitted radiation lacks the intensity, power, irradiance, fluence rate, fluence, effective irradiance, or total irradiance necessary for inactivating pathogens on objects or surfaces in the treatment space.

The far-UV apparatus can be configured, or include instructions for deployment, such that it provides sufficient irradiation to inactivate pathogens in air, but without providing the intensity, power, irradiance, fluence rate, fluence, effective irradiance, or total irradiance necessary for effective inactivation of pathogens on an occupant, occupant skin, or other surface, object, or surface in the treatment space objects or surfaces in the treatment space

In various aspects, the far-UV apparatus advantageously includes a stand, mount, or instructions, such that when deployed, the apparatus will be sufficiently far from expected locations of occupants so as to attenuate the intensity, power, irradiance, fluence rate, fluence, effective irradiance, or total irradiance so as to avoid effective germicidal action of an occupant, occupant skin, or other surface, object, or surface in the treatment space.

In various aspects, the far-UV apparatus is configured with an optical component so as to sufficiently diffuse emitted light or broaden the broadcast pattern so as to attenuate the intensity, power, irradiance, fluence rate, fluence, effective irradiance, or total irradiance so as to avoid effective germicidal action of an occupant, occupant skin, or other surface, object, or surface in the treatment space. In various aspects, the far-UV apparatus is configured with an optical component to transmit one or more wavelength of far-UV radiation and reflect one or more wavelength of visible light. In various aspects, the far-UV apparatus is configured with an optical component to transmit a majority of radiation at 222 nm, and reflect a majority of radiation at one or more wavelength of visible light. In various aspects, the far-UV apparatus is configured with an optical component to transmit a majority of radiation at 222 nm from the far-UV radiation source to the space being irradiated, and reflect a majority of visible light at 500 nm, 550 nm, 600 nm, 650 nm, 700 nm, or a combination thereof, from the space being irradiated back to the space being irradiated. In various aspects, the far-UV apparatus is configured with an optical component to transmit a majority of radiation at 222 nm, and reflect a majority of light at 500-700 nm from the space being irradiated back to the space being irradiated. In various aspects, the far-UV apparatus is configured with an optical component to transmit far-UV radiation and reflect blue, green, yellow, orange, or red light, or a combination thereof, from the space being irradiated back to the space being irradiated.

In various aspects, the far-UV apparatus is configured to pulls air towards it in a manner that avoids generating counter rotating vortices, air walls, or other forms of air flow that partition the air space. The apparatus can avoid generating counter rotating vortices due to configuration of its air intake and air exhaust, or based on its deployment within a room. In various aspects, the apparatus provides the advantage of viral movement within the occupiable space.

The apparatus of the present disclosure can be configured as a portable unit. For example, the apparatus can be configured with one or more handles. The disclosure also providers a kit that comprising the apparatus as part of a portable system. The apparatus, or kit, can include a stand, such as a floor stand or tabletop stand, a wall mount, or a ceiling mount. The stand can be a telescoping or collapsible stand. In various examples, the stand configurable to a height of at least 4 feet. In some examples, the apparatus can be configured for attaching the air stream housing to a return or a supply of a pre-existing HVAC system. In various examples, the apparatus, or kit, is 60 lbs. or less and has a volume of 20 cu ft or less. For example, the apparatus, or kit, can be about or less than 10 lbs., 20 lbs., 30 lbs., 40 lbs., 50 lbs., or 60 lbs. The apparatus, or kit, when deployed, can be about 1 cu ft or less, 2 cu ft or less, 3 cu ft or less, 4 cu ft or less, 5 cu ft, 10 cu ft, 15 cu ft, or 20 cu ft. In various examples, the apparatus has a footprint of less than 1-5% of the space for which it is configured to treat. In some examples, the apparatus has a footprint of less than 1 sqft, 2 sqft, 3 sqft, 4 sqft, or 5 sqft. The apparatus, or kit, when deployed or disassembled for transport, can have height, width, or length, if present, of 10 inches or less, 20 inches or less, 30 inches or less, or 40 inches or less. In various examples, each of the height, width, or length, if present, of 20 inches or less, 30 inches or less, or 40 inches or less. In some aspects, the apparatus includes a cap or a hinged cover. In yet further aspects, the apparatus can include an external reflector or diffusor. In alternative aspects, the apparatus can be instead configured as a permanent or semi-permanent fixture.

### EXAMPLES

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter. Aspects of the present disclosure can be realized by various examples, some of which are provided in the drawings, but a person skilled in the art would readily appreciate that the specific examples provided herein are neither limiting nor exhaustive, and that any number of additional variations can be immediately envisaged as falling within the spirit of the invention.

Indeed, the various examples and drawings illustrated herein can include elements in addition to those shown or described. However, the present inventors also contemplate examples in which only those elements shown or described are provided. Moreover, the present inventors also contemplate examples using combinations of those elements shown or described, either with respect to a given example, or with respect to other examples shown or described herein. Use of headers in this document is not intended to limit the subject matter in any way, and it is further contemplated that the various aspects discussed with respect to one particular illustrated example can be envisaged with respect to each other.

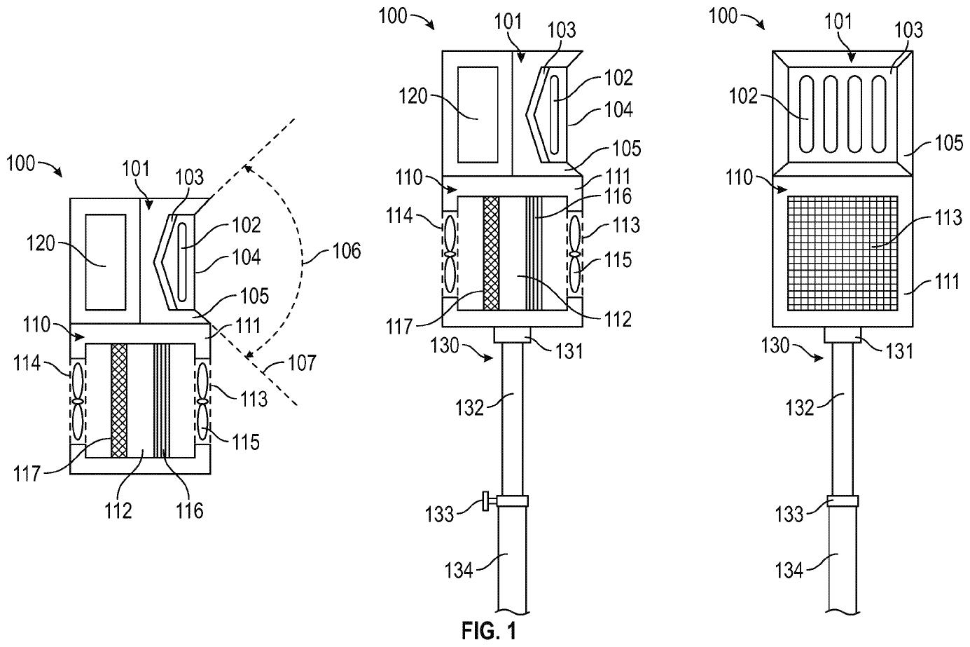

FIG. 1 provides a schematic illustration of an example far-UV apparatus 100 having a far-UV radiation source 101 externally integrated with an air stream unit 110. FIG. 1 illustrates one example of a far-UV radiation source 101, which includes a include a bulb 102, a reflector 103, and lens 104, which are contained within a lamp housing 105. The lamp housing 105 provides a cavity where the bulb 102 is secured and protected. The reflector 103 serves to reflect light away from the inside of the lamp housing out through the lens 104. In various aspects, the lens 104 can include an optical filter. As such, the lens 104 can serve the functional of filtering various wavelengths from emitted radiation. The lens 104 can, but need not necessarily, magnify, focus, or disperse light. The far-UV radiation source 101 emits light in a broadcast pattern, which is illustrated in FIG. 1 as having a broadcast angle 106 and a broadcast boundary 107. The lamp housing 105 can be configured to protect or recess the lamp while substantially avoiding blocking the transmittance of any emitted light. In another aspect, the lamp can be flush with the housing.

FIG. 1 illustrates one example of an air stream unit 110. The air stream unit 110 includes an air stream housing 111, which provides a path for an air stream and a means for generating air flow via a fan system of one or more fans 115. The air stream housing 111 defines an air flow chamber 112, an air intake 113, and an air exhaust 114. The area between the air intake 113 and air exhaust 114 defines an air flow chamber 112 that provides a space for one or more fans 115 of the fan system, one or more mechanical filter 116, or one or more scrubber 117. The fans 115, mechanical filter 116, or scrubber 117 can be mounted throughout the air flow chamber 112, including mounting near the air intake 113 or air exhaust 114. As illustrated in the particular example of FIG. 1, fans 115 are positioned the air intake 113 and air exhaust 114 and secured to the air stream housing 111, and a mechanical filter 116 is placed in series before a scrubber 117 in internal cavity of the air flow chamber 112. The fans 115 are configured to provide air flow from the air intake 113 through the air flow chamber 112 to the air exhaust 114. In various aspects, the locations of the air intake 113 and air exhaust 114 can be reversed so as to provide air flow in the opposite direction.

The air stream unit is integrated with the far-UV radiation source 101 such that the far-UV source 101 is mounted upon an exterior surface of the air stream unit 110 and thus outside of the air flow chamber 112. As illustrated in the particular example of FIG. 1, the far-UV radiation source 101 and the air stream unit 110 can share a surface. A power supply 120 provides power to the components of the far-UV radiation source 101 and the air stream unit 110. The far-UV radiation source 101 is positioned adjacent to air intake 113 and oriented to emit radiation away from the components of the far-UV apparatus 100. As illustrated in the particular example of FIG. 1, the air stream unit 110 is configured to capture irradiated air from in front of the far-UV light source and to direct it through the air stream housing 111 and out of the air exhaust 114.

The far-UV apparatus 100 can include an adjustable support 130, which is secured to the integrated far-UV radiation source 101 and air stream unit 110 via an adjustable support 130. The adjustable support 130 can include a telescoping pole 132, adjustment knob and vice 133, and a pole retainer 134. The adjustable support 130 can be a collapsible, telescoping tripod stand. The air stream unit 110 and the far-UV radiation source 101 can be configured to approximately share a dimension, e.g., width, and the air intake can be configured to approximately share a dimension with the reflector 103. In various aspects, the air stream unit 110, the far-UV radiation source, or both, can be cuboid.

The air stream unit 110 is configured to capture irradiated air from in front of the far-UV light source and to direct it through the air stream housing 111 and out of the air exhaust 114. Alternatively, the air stream unit 110 can be configured to exhaust air into the area of irradiated area in front of the far-UV light source. The far-UV apparatus 100 when mounted on the stand can conveniently provide an air stream through irradiated air space at, slightly above, or slightly below, the height of a human. In other aspects, the far-UV apparatus 100 includes a stand such that the air stream is provided approximately 2, 3, 4, 5, 6, 7, 8, 9, 10, 11, 12, 13, 14, 15, 16, 17, 18, 19, or 20 feet off the ground.

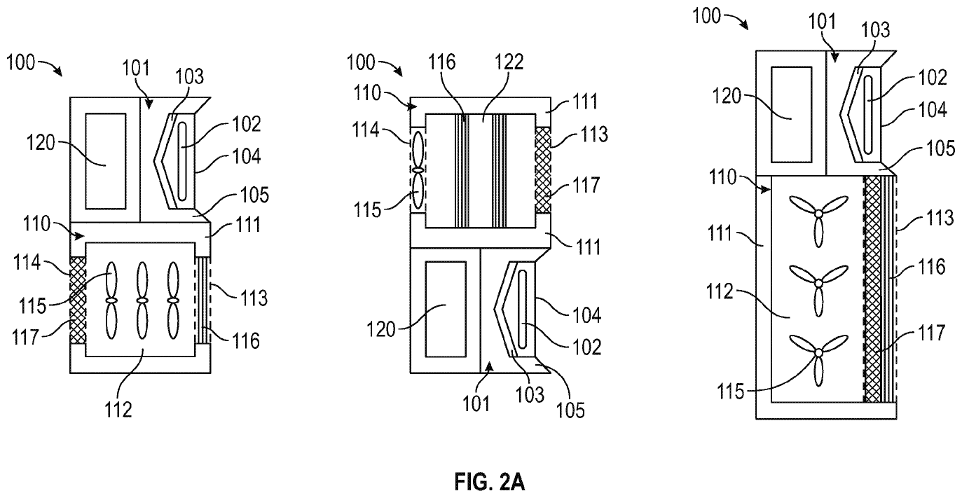

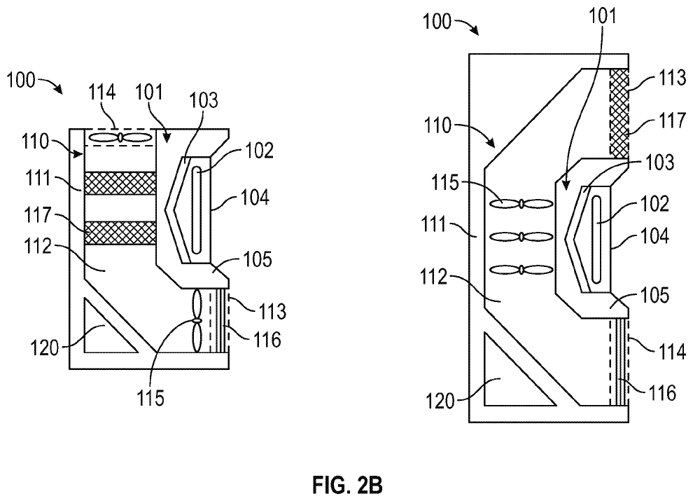

FIG. 2, including FIG. 2A and FIG. 2B, provides schematic views of various examples of a far-UV apparatus having a radiation source integrated with an air stream apparatus. In each example, a far-UV apparatus 100 having a far-UV radiation source 101 is externally integrated with an air stream unit 110. The far-UV radiation source 101 includes a bulb 102, a reflector 103, and lens 104, which are contained within a lamp housing 105. The air stream unit 110 includes an air stream housing 111, which provides a path for an air stream and a means for generating air flow via a fan system of one or more fans 115. The air stream housing 111 defines an air flow chamber 112, an air intake 113, and an air exhaust 114, and the one or more fans 115 provides air flow therethrough. The air flow chamber 112 provides space for one or more fans 115 of the fan system, one or more mechanical filter 116, or one or more scrubber 117. The fans 115, mechanical filter 116, or scrubber 117 can be mounted throughout the air flow chamber 112, including mounting near the air intake 113 or air exhaust 114.

As illustrated in FIG. 2, the far-UV apparatus 100 can have a number of different configurations with respect to location of the air intake 113, air exhaust, far-UV radiation source 101, and air stream housing 111. For example, the far-UV radiation source 101 can above, below, or to the side of, the air stream housing 111, where such directions correspond to how the apparatus 100 would be positioned on a surface, stand or mount 131. As another example, the air intake 113, air exhaust 114, or far-UV apparatus 100 can be located above, below, or to the side of, the far-UV radiation source 101, or any combination thereof. In some aspects, the air intake 113 and air exhaust 114 are configured to flow air from the front of the apparatus 100 to back, from the back to the front, from the front to the top, from the front to one or more side, from the back to the top, from the back to one or more side, from one or more side to the front, from one or more side to the back, from one or more side to the top, or any combination thereof. The far-UV apparatus 100 can also be configured to flow air in and out of the same side. For example, the far-UV apparatus 100 can be configured to flow air in through an air intake 113 on the front of the far-UV apparatus 100 and out through an air exhaust 114 that is also on the front. As another example, the far-UV apparatus 100 can be configured to pull air in through an air intake 113 below the far-UV radiation source 101 and exhaust air above the far-UV radiation source 101; pull air in through an air intake 113 above the far-UV radiation source 101 and exhaust air below the far-UV radiation source 101; pull air in through an air intake on either side of the front of the far-UV apparatus 100 and exhaust air above and/or below the far-UV radiation source 101; or pull air in through an air intake 113 on the front of the apparatus above and/or below the far-UV radiation source 101, and exhaust air on either side of the front of the far-UV radiation source 101. In yet further examples, the apparatus can comprise multiple air intakes air intake 113, multiple air exhausts air exhaust 114, or both. In some examples, the far-UV apparatus 100 comprises two or more air intakes 113, or two or more air exhausts 114, that flank the far-UV radiation source. In further examples, the apparatus comprises two or more air intakes 113, or air exhausts 114, on either side of the far-UV apparatus 100.

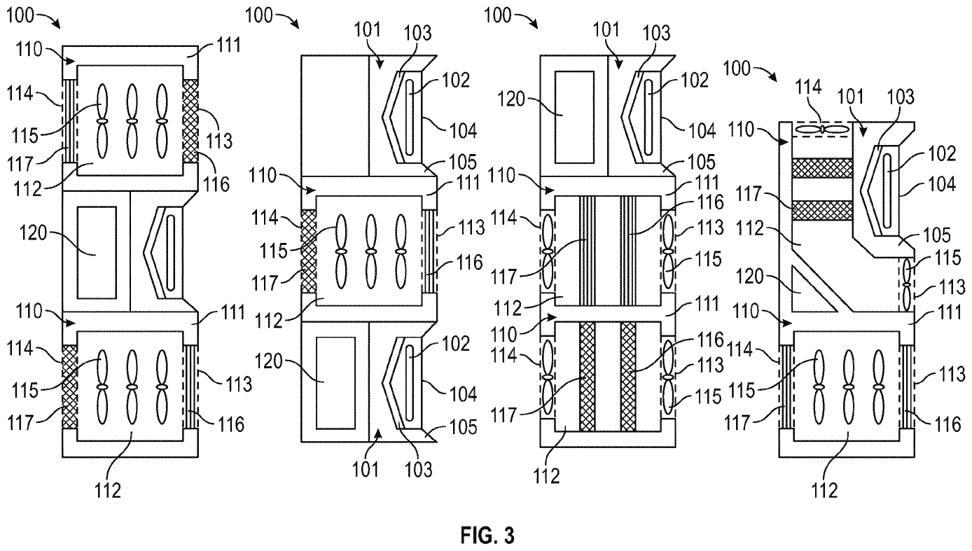

FIG. 3 is a schematic of various further examples of an externally far-UV apparatus 100. The various illustrated examples utilize one or more far-UV radiation source 101 externally integrated with one or more air stream housing 111, which are all together integrated into a single far-UV apparatus 100. The various components can be integrated together by becoming physically connected along one or more surface, by sharing a single power source, by being mounted to a shared support, or by otherwise being physically co-located and fixed together. For example, the far-UV apparatus 100 can include two or more air stream housings 111 integrated with a single far-UV radiation source 101. The apparatus can include one air stream housing 111 integrated with two or more far-UV radiation sources 101. The various subcomponents of each far-UV radiation source 101 and air stream housing 111 can be the same or differ. For example, the apparatus can comprise a first air stream housing 111 and a second air stream housing 111. In a further example, the first air stream housing can comprise a mechanical filter 116, while the second air stream housing can comprise a scrubber 117. The two or more air stream housings 111 can provide two or more air streams, such that the air streams can be in the same direction, opposite directions, or otherwise directed to capture irradiated air or to exhaust purified air to the targeted space. The first air stream housing 111 and the second air stream housing 111 can be integrated together by becoming physically connected along one or more surface, by sharing a single power source, by being mounted to a shared support, or by otherwise being physically co-located and fixed together. As another example, the far-UV apparatus 100 can comprise a first far-UV radiation source 101 and a second far-UV radiation source 101. As yet another example, the apparatus can comprise a first far-UV radiation source 101 and a second radiation source that is other than far-UV radiation, such as visible light.

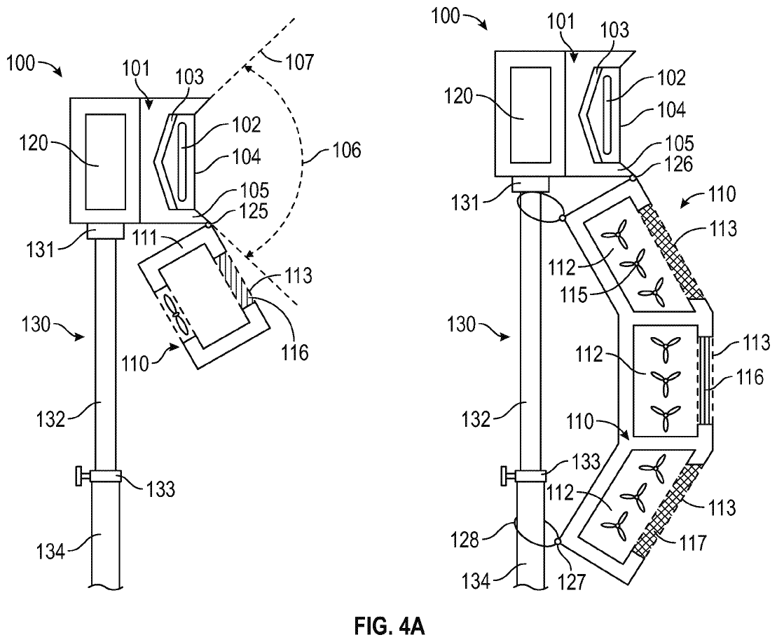

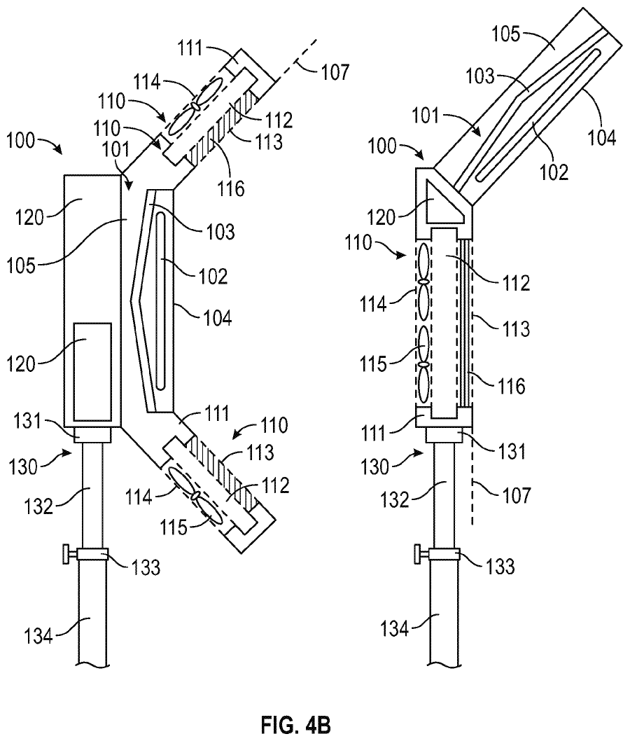

FIG. 4, including FIG. 4A and FIG. 4B, provide schematic views of yet further examples of a far-UV apparatus 100. The far-UV apparatus 100 can include a far-UV radiation source 101 in physical communication with an air stream unit 110. The lamp housing 105 and the mechanical filter air stream housing 111 can be attached together via an attachment point 125. The attachment point 125 can be a fastener, hinge, or reversible coupling. At least a portion of the air stream unit 110 and air stream housing 111 is configured to be positioned close to the broadcast pattern and approximate a broadcast boundary 107 of the broadcast pattern of the far-UV radiation source. At least one of the air intakes 113 and/or air exhaust 114 can be configured to be positioned close to the broadcast pattern and approximate a broadcast boundary 107 of the broadcast pattern of the far-UV radiation source. For example, at least a portion of the air stream unit 110, air stream housing 111, air intake 113, and/or air exhaust 114 can be configured within or about 0.5 cm, 1 cm, 2 cm, 3 cm, 4 cm, 5 cm, 6 cm, 7 cm, 8 cm, 9 cm, or 10 cm, of the broadcast boundary 107. As another example, at least a portion of the air stream unit 110, air stream housing 111, air intake 113, and/or air exhaust 114 can be configured having a surface at an angle that is less than or about 0°, 5°, 10°, 15°, 20°, 25°, 30°, 35°, 40°, 45°, or 50° from the broadcast boundary 107.

The far-UV apparatus 100 can be further configured having a first air stream unit 110 positioned along the broadcast boundary 107, and yet contain one or more auxiliary components having one or more air stream unit 110, air flow chamber 112, air intake 113, positioned further away. In various aspects, a portion of the air stream unit 110 and/or air stream housing 111 can be coupled to a portion of the adjustable support 130 via a lower attachment point 127 and a coupler 128. The far-UV apparatus 100 can also be configured so that two or more air stream units 110 are positioned on opposing sides of the far-UV radiation source 101 along different broadcast boundaries 107.

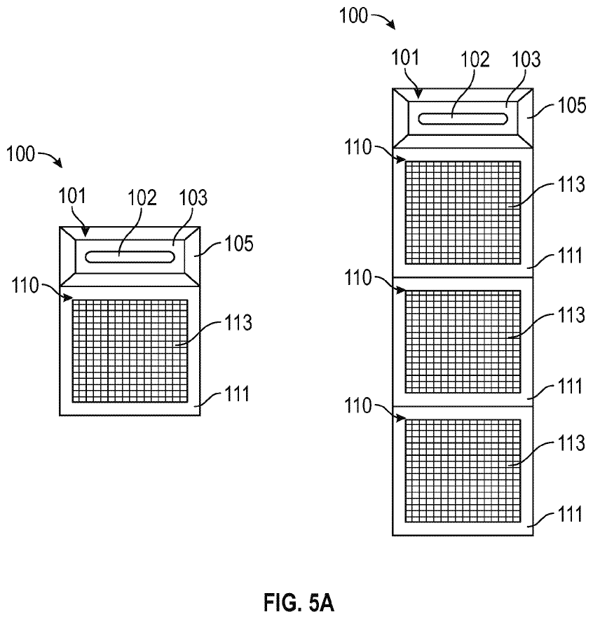

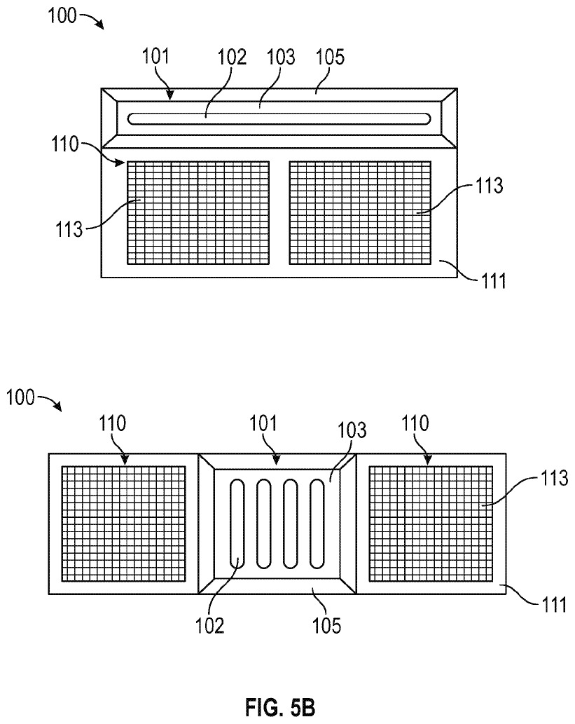

FIG. 5, including FIG. 5A and FIG. 5B, illustrates a far-UV apparatus having an air intake 113 on the same face from which the far-UV radiation source 101 emits radiation. In other aspects, one or more air intake 113 is replaced with an air exhaust 114. In various aspects, the far-UV apparatus 100 can have a far-UV radiation source 101 and an air stream unit 110 having at least one dimension that is substantially shared. In yet further aspects, the far-UV apparatus 100 can have air intake 113 and/or air exhaust 114 that shares at least one dimension with the bulb 102, reflector 103, or lens 104. In further aspects, the far-UV apparatus 100 can have air intake 113 and/or air exhaust 114 that shares at least one dimension with the combined width or heigh of an assembly of bulbs 102.

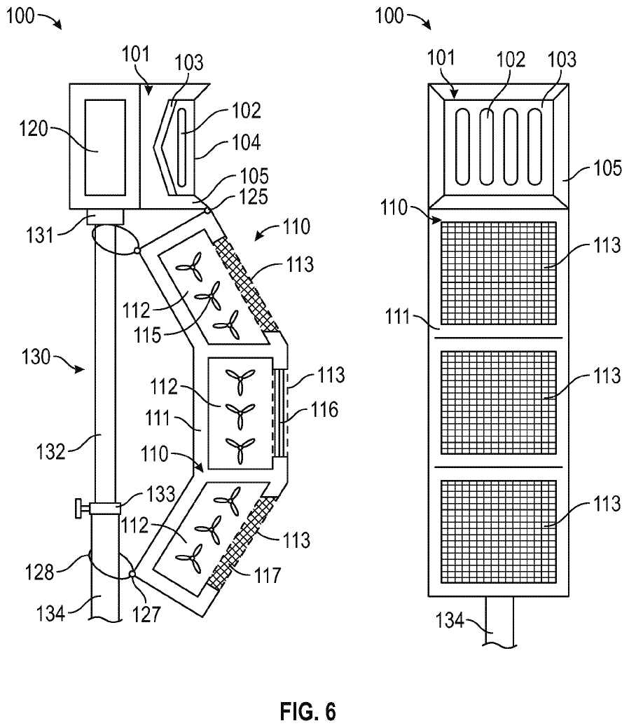

FIG. 6 is an example of a far-UV apparatus 100 having multiple air flow chambers 112 that are physically attached to the far-UV radiation source 101 via attachment point 125. The attachment point 125 can control the angle at which the closest air intake 113 approaches broadcast boundary 107 of the emitted radiation. This configuration permits the air stream unit 110 to be close to the area of irradiated air that has highest intensity of radiation, yet avoid absorbing or blocking the radiation from reaching the rest of the space being treated. The adjustable support 130 provides a means for elevating and positioning the far-UV radiation source 101 and air stream unit 110. For example, it can be positioned to provide irradiation and/or air movement at about head height. The adjustable support 130 can also facilitate positioning the far-UV radiation source 101 above and out of reach of occupants to avoid tampering or to provide a minimum distance between occupants and the far-UV radiation source 101. The adjustable support 130 also provides a simply way for a use to control the direction of radiation and air movement. Moreover, the adjustable support 130 also provides additional support for the one or more air stream units 110 or a large air stream unit 110. In some aspects, the one or more far-UV radiation source 101, air stream unit 110, and adjustable support 130 are provided to the user modularly, as separate components, with instructions to combine them as presently described. The multiple air flow chambers 112 and air intakes 113 can be configured having a single air stream housing 111 defining multiple air flow chambers 112, or it can be configured having multiple air stream housings 111 that are physically attached together, such as by sharing one or more wall of respective, separate air stream housings 111.

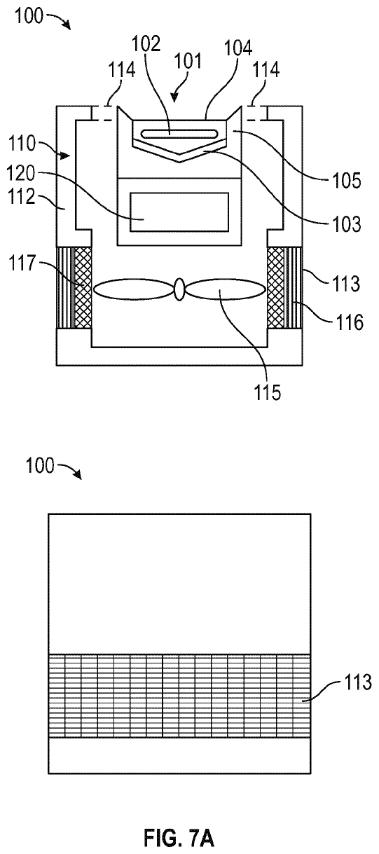

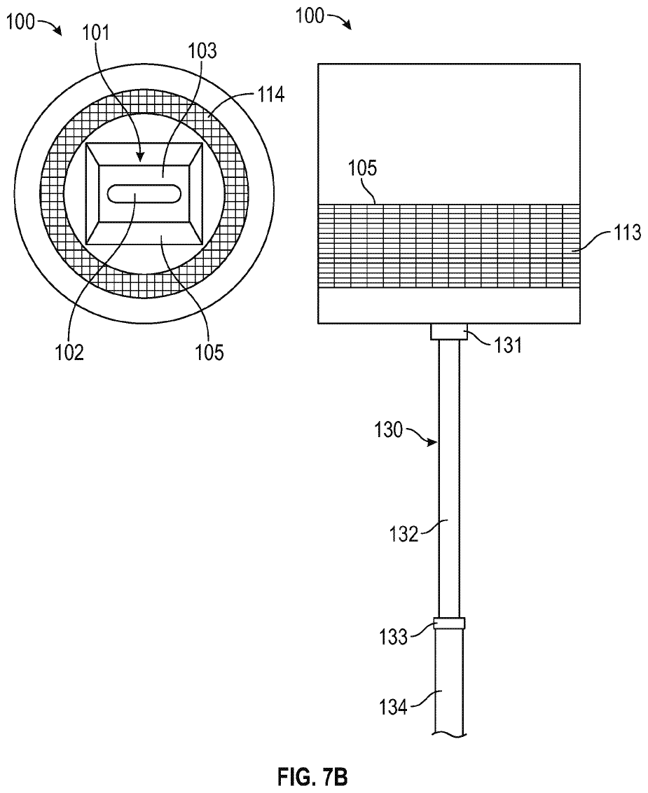

FIG. 7, including FIG. 7A and FIG. 7B, provide an example of a far-UV apparatus 100 that provides an airstream around a far-UV radiation source 101. The far-UV apparatus 100 can be configured to have a cylinder shape, by way of a cylindrical air stream housing 111. The far-UV apparatus 100 can be configured to exhaust air around the far-UV radiation source 101 or it can be configured for intake of air around the far-UV radiation source 101. The far-UV radiation source 101 has a lamp housing 105 containing at least one bulb 102, a reflector 103, and a lens 104. The bulb 102 emits far-UV radiation through the lens 104 away from the far-UV apparatus 100. The far-UV radiation source 101 is encircled by an air stream housing 111. The air stream housing 111 provides an air flow chamber 112, air intake 113, and air exhaust 114. One or more fans 115 provide an air stream that travels through the air intake 113, the air flow chamber 112, and the air exhaust 114. The provided air stream serves to move irradiated air.

As illustrated in FIG. 7, a circumferential, or partially circumferential, air intake 113 accepts air into an air flow chamber 112. The air flow chamber 112 is equipped with one or more fans 115, which pulls captured air over one or more mechanical filter 116 and/or scrubber 117. The air stream housing air stream housing 111 can be configured to accept a cylindrical mechanical filter 116, a cylindrical scrubber 117, or both. The air stream housing 111 can also be configured to direct air flow from the sides to the top, from the top to the sides, from the top through the bottom, or from the bottom through the top. The base of the air stream housing 111 can be closed. The power supply 120 can be positioned centrally within the air stream housing 111, such as along the axis below the far-UV radiation source 101 and above the one or more fans 115. The power supply 120 powers at least the far-UV radiation source 101 and the one or more fans 115. A support structure (not shown) can provide support for both the one or more fans 115, the power supply 120 and the far-UV radiation source 101. In some aspects, the far-UV radiation source 101 can be configured such that the air stream provides cooling for the power supply 120 and the far-UV radiation source 101.

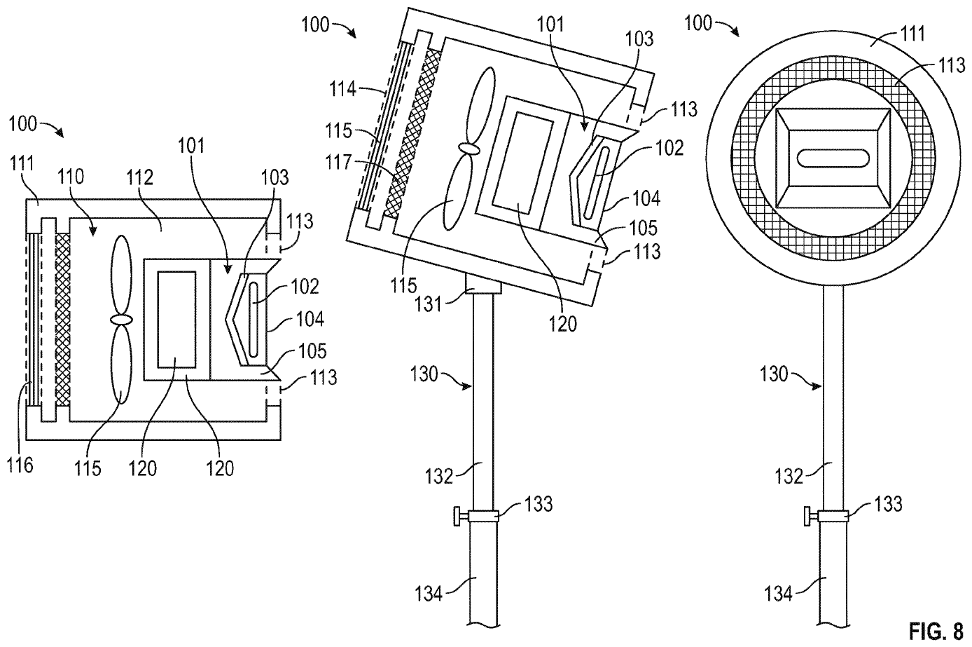

As illustrated in FIG. 8, the far-UV radiation source 101 is directed away from the rest of the far-UV apparatus 100 and its components. A circular air exhaust 114 exhausts air from the air stream. In some aspects, the air stream provided by the air exhaust 114 can be configured to flow across the surface of the far-UV radiation source 101 and lens 104, and across the irradiated air space. In some aspects, the air stream provided by the air exhaust 114 can be configured to provide clean, filtered air to the proximate irradiated air space. In some aspects, the air stream provided by the air exhaust 114 can be configured exhaust to the sides, away from the far-UV radiation source 101, in a manner which pulls irradiated air away from the irradiated air space or provides a vortex. The integrated air stream housing 111 can be configured having an adjustable support 130. When raised and mounted on the adjustable support 130, the far-UV apparatus 100 can serve to move air around and between occupants at head level.

In an alternative aspect, the far-UV apparatus 100 depicted in FIG. 8 can be configured such that the position of the air intake 113 and air exhaust 114 are switched. In such configuration, the one or more fans 115 are configured to direct the air stream in an opposite direction. For example, the far-UV apparatus 100 can be configured such that it captures irradiated air from in front of the far-UV radiation source 101, and the air stream exhausts through the side. In such configuration, the far-UV apparatus 100 filters the irradiated air.

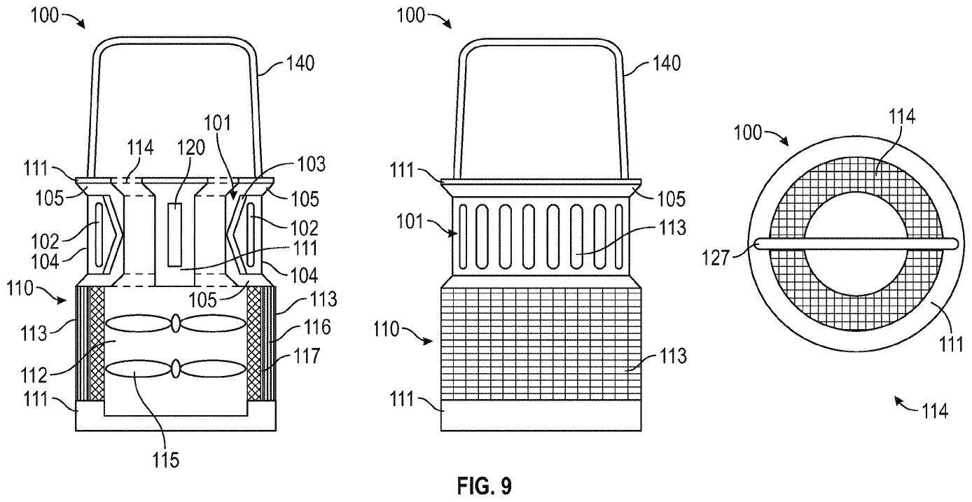

FIG. 8 is another example of a far-UV apparatus 100 that provides an airstream around a far-UV radiation source 101. The far-UV apparatus 100 of FIG. 9 has a cylindrical form with an open base and closed walls. Such configuration can be mounted via mount 131, which can be adjustable to control pitch, on a cylindrical side wall of the air stream housing 111. Moreover, because the cylindrical base is open, the far-UV apparatus 100 can be oriented to direct an air stream according to the needs of a use or as dictated by a treatment space. For example, in various aspects, the far-UV apparatus 100 can be raised and mounted an adjustable support 130 and set at a pitch that provides an air flow through irradiated and occupied air.

As illustrated in FIG. 8, the far-UV radiation source 101 is directed away from the rest of the far-UV apparatus 100 and its components. A circular air exhaust 114 captures irradiated air and provides an air stream. Thus, in some aspects, the air stream provided by the air intake 113 can be configured to move and filter irradiated air. The air exhaust 114 can be configured to direct exhausted air in a manner which serves to increase movement of air through the room, particular air at head-level around occupants in a treatment space.

In an alternative aspect, the far-UV apparatus 100 depicted in FIG. 8 can be configured such that the position of the air intake 113 and air exhaust 114 are switched. In such configuration, the one or more fans 115 are configured to direct the air stream in an opposite direction. For example, the far-UV apparatus 100 can be configured to provide filtered and/or scrubbed air to the space in front of the far-UV radiation source 101. In such configuration the air subject to highest intensity radiation is filtered and/or scrubbed air.

FIG. 9 is another of an integrated far-UV air stream apparatus 100, which can have a handle 140 that facilitates portability.

As illustrated in FIG. 9, the far-UV apparatus 100 can include a plurality of far-UV radiation sources 101. The far-UV radiation sources 101 can be arranged circumferentially on the outer surface of the far-UV apparatus 100 to provide a cylindrical shape. The far-UV radiation sources 101 each have a lamp housing 105 that holds the several bulbs 102, reflectors 103, and one or more lens 104. The far-UV radiation sources 101 are positioned external to an air stream unit 110, which occupies the bottom and inside of the far-UV apparatus 100. The air stream unit 110 comprises an air stream housing 111, which defines the air flow chamber 112, air intake 113 and air exhaust 114. One or more fans 115 provide an air stream that travels through the air intake 113, the air flow chamber 112, and the air exhaust 114. The provided air stream serves to move irradiated air.

The air stream housing 111 can also be configured to direct air flow from the sides to the top, or from the top to the sides. The base of the air stream housing 111 can be closed. The power supply 120 can be positioned centrally within the air stream housing 111, such as between the plurality of far-UV radiation sources 101 and above the one or more fans 115. The power supply 120 powers at least the far-UV radiation source 101 and the one or more fans 115. A support structure (not shown) can provide support for both the one or more fans 115 and the power supply 120. In some aspects, the far-UV radiation source 101 can be configured such that the air stream provides cooling for the power supply 120 and the far-UV radiation source 101.

The air stream housing 111 and air intake 113 can be configured to capture irradiated air near the far-UV radiation sources 101. The captured irradiated air can be passed through one or more mechanical filter 116, one or more scrubber 117, or both.

The air stream housing 111 and air exhaust 114 can be configured to exhaust the air stream upwards, toward the irradiated air, or both. For example, the air stream housing 111 can be configured so as to deflect exhausted, purified air back toward the area of irradiated air proximate to the far-UV radiation sources 101 and subject to high intensity irradiation.

The far-UV apparatus 100 having a handle can be useful for both portability and for positioning the apparatus. The far-UV apparatus 100 can be positioned amongst groups of people, such as on tables, countertops, spectator seating areas, retail checkouts, and in other crowded spaces where air in the vicinity around people can be difficult to disrupt. The air movement provided by operation of the apparatus can serve to disrupt and move the air near, around, and between people thus permitting greater exposure to the provided radiation and thwarting short term transmission.

In some aspects, the handle is reflective to emitted radiation on a portion of the handle's surface that faces the far-UV radiation source 101.

In an alternative aspect, the far-UV apparatus 100 depicted in FIG. 9 can be configured such that the position of the air intake 113 and air exhaust 114 are switched. In such configuration, the one or more fans 115 are configured to direct the air stream in an opposite direction.

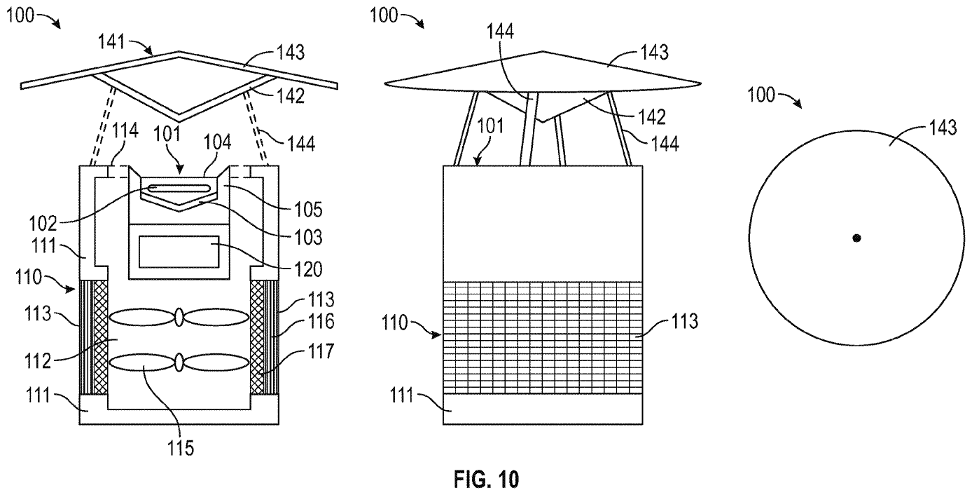

FIG. 10 is yet another of an integrated far-UV air stream apparatus 100, which can have an external reflector 141.

The external reflector 141 can redirect radiation emitted from the far-UV radiation source 101 so as to broaden the irradiated air space. The external reflector 141 can be configured to reflect radiation away from the far-UV apparatus 100 and its components.

The external reflector 141 can include a far-UV reflective surface 142, one or more supports 144, and a cap 143. The bottom of the cap 143 can provide a far-UV reflective surface 142. The cap 143 can also be useful for outdoors use, serving to prevent rain or debris from fouling the apparatus. In some aspects, the cap 143 can serve to shield an occupant from a direct radiation from the far-UV radiation source 101. The external reflectors 141 can be made of a far-UV reflective material, such as TEFLON® or other PTFE material.

As illustrated in FIG. 10, the far-UV radiation source 101 can be oriented to emit radiation upwards and the external reflector 141 can be positioned above the far-UV radiation source 101. This configuration can be useful when positioning the far-UV apparatus 100 above the air intended to be irradiated, such as when raised on an adjustable support 130 and elevated overhead in a room.

The external reflector 141 can also serve to shape the movement of air provided by the air stream unit 110. For example, as illustrated in FIG. 10, the air stream unit 110 can emit filtered and/or scrubbed air through the air exhaust 114. The filtered, exhausted air is redirected outwards so a greater amount of irradiated air proximate to the far-UV radiation source 101 is filtered irradiated air. In such a configuration, the cap 143 can also be useful when deployed in an occupiable space lacking ceilings, such as outdoors, as it can facilitate air movement through the treatment space. It can also serve to disperse the exhausted air, particularly in a 360-degree pattern.

The external reflector 141 is configured to reflect at least a portion of the radiation broadcasted from the far-UV radiation source 101, but in various aspects the external reflector 141 can be configured to permit some amount of radiation to emit upwards. The air stream unit 110 comprises an air stream housing 111, which defines the air flow chamber 112, air intake 113 and air exhaust 114. One or more fans 115 provide an air stream that travels through the air intake 113, the air flow chamber 112, the air exhaust 114, and deflected by the external reflector 141. The provided air stream serves to move irradiated air away from the area of greatest intensity of radiation and downward for ultimate recapture by an air intake 113 powered by the one or more fans 115 and power supply 120.

In an alternative aspect, the far-UV apparatus 100 depicted in FIG. 10 can be configured such that the position of the air intake 113 and air exhaust 114 are switched. In such configuration, the one or more fans 115 are configured to direct the air stream in an opposite direction. In one example, the far-UV apparatus 100 is configured so that irradiated air is sucked into the air stream unit 110 and emitted from the sides. Such configuration thus filters and/or scrubs irradiated air.

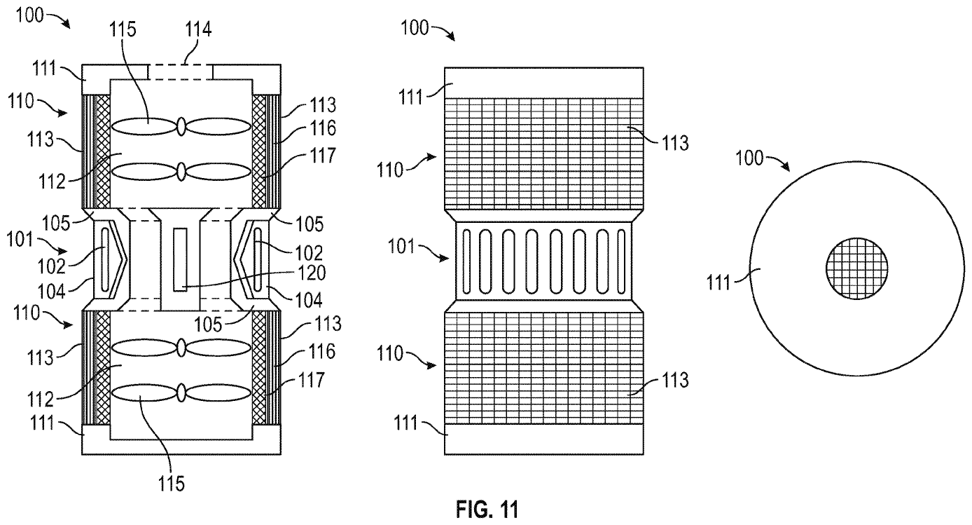

FIG. 11 is another example of an integrated far-UV air stream apparatus 100, which contains two or more air intakes 113 and having the far-UV radiation source 101 positioned between them.

As illustrated in FIG. 11, the far-UV apparatus 100 can have a substantially cylindrical shape with a circumferential far-UV radiation source 101 around an air stream unit 110 that provides air intakes 113 both above and below the far-UV radiation source 101. The air stream unit 110 has an air stream housing 111, which defines the air flow chamber 112, air intakes 113, and an air exhaust 114. One or more fans 115 provide an air stream that captures irradiated air through the air intakes 113, sends the captured irradiated air through the air flow chamber 112, and out of the air exhaust 114. The provided air stream serves to move irradiated air. The air intakes 113 can capture irradiated air both above and below the far-UV radiation source 101. The base of the cylindrical air stream housing 111 can be closed to provide a surface to rest the far-UV apparatus 100. The single air exhaust 114 can provide directed air circulation. For example, the air exhaust 114 can direct the exhausted air toward the ceiling of a room where it deflects and facilitates air circulation.

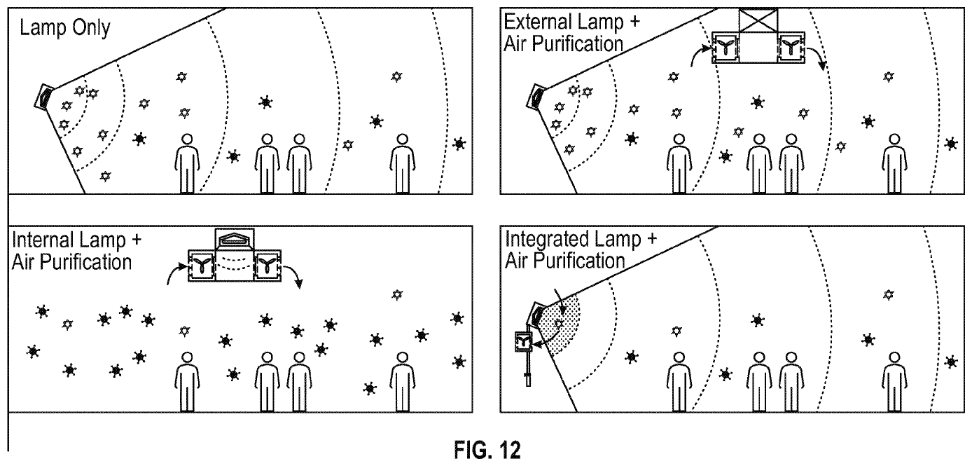

In an alternative aspect, the far-UV apparatus 100 depicted in FIG. 12 can be configured such that air intake 113 and air exhaust 114 are switched. In such configuration, the one or more fans 115 are configured to direct the air stream in an opposite direction. For example, the far-UV apparatus 100 can be configured to provide filtered and/or scrubbed air into the irradiated space proximate to the far-UV radiation source 101 that is subject to high intensity radiation.

FIG. 12 provides a chart comparing the far-UV apparatus 100 with other approaches to far-UV disinfection.

A far-UV lamp alone is capable of inactivating pathogens in air, but can generate certain hazardous active species due to the action of far-UV light on oxygen, small organic molecules, and airborne particulate matter. Additionally, some airborne pathogens can avoid far-UV inactivation by residing behind obstacles, people, or simply outside the broadcast pattern of the far-UV lamp.

Separate air filters, such as the air filters in HVAC systems, can filter hazardous species from the air, but typically do so by utilizing low penetrating filters and powerful fans to move air through such filters. Such systems, especially when configured for filtration, can fail to provide sufficient air movement to facilitate far-UV based inactivation of pathogens in an occupiable space.

Duct-based solutions and UV sterilization chambers can be poorly suited for both inactivating airborne pathogens and removing hazardous active species from air in an occupiable space. UV ducts and sterilization chambers rely on fairly high residence time for pathogens within the far-UV treatment space within the duct or sterilization chamber. The high residence time requires slower airflow through such internal treatment space, which in turn provides fewer air exchanges and poor movement of air in the occupiable space. Such solutions have a problem when seeking to address both airborne pathogens and hazardous active species.

The far-UV apparatus described herein can have advantages arising from decoupling the far-UV treatment from the filtration and/or scrubbing treatment. The integration of the far-UV radiation source and air stream unit provides a proximity effect: the position and orientation of the air stream unit relative to the far-UV radiation source provides air movement in the irradiated space, particularly at the space proximate to the far-UV radiation source that is subject to higher intensity radiation. Following an inverse square relationship, hazardous active species form closer to the far-UV radiation source and such species are thought to arise due to non-pathogenic contaminants in air, such as ozone and ozone-derived products. In various aspects described herein, the far-UV apparatus can reduce hazardous active species by removing non-pathogenic contaminated air from the highly irradiated space proximate to the radiation source, by providing filtered and/or scrubbed air to the highly irradiated proximate to the radiation source, or both. The treatment space can be subject to irradiation for long periods and airborne pathogens residing in the treatment space can be inactivated with lower levels of far-UV radiation owing to longer treatment time. At the same time, the internal filtration and/or scrubbing mechanism can work at high flow to quickly act upon the treatment area that is most susceptible to generating hazardous active species. The air stream permits multipass filtration and/or scrubbing of hazardous active species and their precursors, while airborne pathogens in the human-use space continue to be subjected to far-UV radiation. The higher air flow provided by the air stream unit can also have the further advantage of providing air circulation throughout the treatment space, including the air space in the immediate vicinity of occupants.

### Computational Fluid Dynamics Simulation

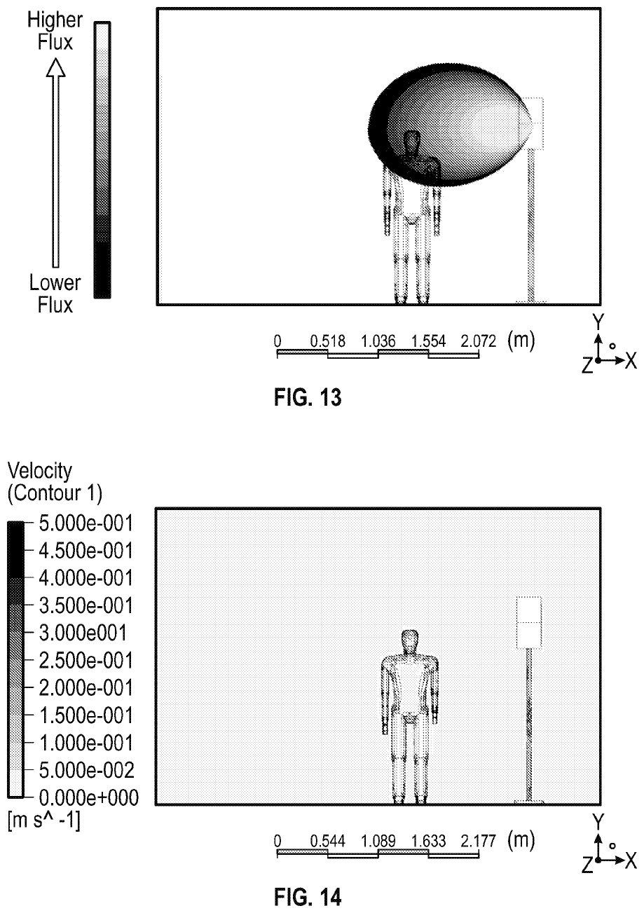

Computer fluid dynamics simulation of particle and gas movement was used to model the effects of various configurations of a germicidal UV apparatus having a far-UV radiation source integrated with an air stream unit. A computational fluid dynamics model of an occupied space with a door was created in which two occupants are located adjacent to a modeled example far-UV apparatus or other far-UV source for comparative examples. The model was generated using SOLIDWORKS computer-aided design software, available from SolidWorks Corp., Dassault Systèmes (Vélizy-villacoublay, France). Meshing and computational fluid dynamics were performed using Ansys CFX available from Ansys, Inc. (Canonsburg, PA). The space was modeled with dimensions of 4.6 m×4.6 m×3.0 m to be fairly representative of typical rooms such as offices, meeting spaces, residential spaces, kitchens, dining rooms, bedrooms, children's spaces, learning areas, and work spaces, with the far-UV apparatus placed 609 mm from the wall in the x direction, and placed in the center of room in the y direction. The modeled example far-UV apparatus was configured having a height of 1869 from the floor to center of the filter and lamp. Boundary conditions for this model included non-slip velocity conditions for all interior surfaces as well as the occupants. Occupants were each placed 1080 mm from the lamp and 1535 mm apart. The door was modeled as an opening with an average static pressure of 0 Pa. A first occupant in the model is represented as a particle generating source of infectious particles (an infected person model), outputting a constant 30 lpm, the second occupant is represented as a non-infected, susceptible individual with an inhalation rate of 30 lpm. Particle emission of the particle generating source of infectious particles is simulated using unit density particles of 1.0 μm diameter with a concentration of 2.0 particles/cm³. The model assumes isothermal conditions, so impacts of thermal buoyancy and temperature were not included. The model mesh consisted primarily of tetrahedral cells, with 159147 nodes and 862256 tetrahedral elements. The far-UV radiation source for each example was configured to provide 0.2 μW/cm², to facilitate comparison between simulations, and which corresponds to a suitable fluence rate for a variety of germicidal UV applications. FIG. 13 provides a fluence rate contour plot illustrating the inverse square relationship between fluence rate and distance from the far-UV radiation source, and identifies exemplary regions of higher fluence rate provided by an example far-UV radiation source.

The modeled example far-UV apparatus was modeled to provide an air stream that passes through a filter in the device. The filter was configured as an approximately 500 mm diameter filter. Several types of filters were tested as representative examples of higher efficiency (lower penetration) to lower efficiency (higher penetration) filters or filter combinations were modeled. These included a HEPA with a filtration efficiency of 99.97%, MERV13 with a filtration efficiency of 60%, and MER V11 with a filtration efficiency of 36%, all configured to provide the same CADR of 375 m³/hr to facilitate comparison. An example CADR of 375 m³/hr was selected as a fairly representative value for CADR levels that may be sought in a portable air treatment device. The filtration efficiency and CADR values corresponded to volumetric flow rates of 375, 625, 1041 m³/hr for the HEPA, MERV13, and MERV11 filters, respectively. Such values can represent either individual filters or series of filters that together achieve such performances. The example far-UV apparatus was also modeled in two operating conditions: a “push” configuration that provides an air stream in an orientation such that the air stream flows from an unirradiated outer area of the space near a wall boundary, through the device, and towards an irradiated interior area of the space near occupants; and a “pull” configuration that provides an air stream in an orientation such that the air stream flows from an irradiated interior area of the space where occupants are located, through the device, and toward an unirradiated outer area of the space near wall boundary. In each case a door was modeled on the far side of the room away from the apparatus on the wall toward which radiation was directed.

As a comparative example, a model was configured where a far-UV radiation device provided no source of air stream in the room. As another comparative example, a model was configured where a far-UV radiation device provided no flow and the space additionally included a separate, discrete device that provided an air stream across the occupied space away from the far-UV radiation device, in the corner away from both the door and the radiation source 150 mm from each wall surface of the corner. The separated air stream generating device was configured such that it provided 375 m³/hr CADR and a volumetric flow rate of 375 m³/hr, and thus corresponding to a HEPA.

The model was used to model particle trajectories from an infectious particle source were modeled. CADR and volumetric flow was derived from filtration efficiency, which is defined according to industry standards. Particle trajectories were tracked to determine room average air velocity, average travel distance time, average travel time, fraction of infectious particles that survive UV treatment before to 1^{st} pass through the filter, fraction of infectious particles that survive after 1^{st} pass through UV treatment and filter. See, Table 1.

TABLE 1

| | | | | | CFD | | | | |
|---|---|---|---|---|---|---|---|---|---|
| | | | | | Room | | | | |
| | Filter | | | | Avg | | | | |
| | Type and | | | | Air | Avg | Avg. | | |
| | Air | Filtration | | | Velocity | Travel | Travel | UV | 1^{st} Pass |
| UV Source | Stream | Efficiency | CADR | Vol. Flow | (m/sec, | Distance | Time | Survival | Survival |
| (μW/cm²) | Direction | (%) | (m³/hr) | (m³/hr) | vol avg) | (m) | (s) | Fraction | Fraction |
| 0.2 | None | N/A | N/A | N/A | 0.010 | 88.7 | 535.8 | N/A | N/A |
| 0.2 | HEPA - | 99.97 | 375 | 375 | 0.021 | 7.71 | 106.9 | 1E−4 | 3E−08 |
| | Pull | | | | | | | | |
| 0.2 | MERV | 60 | 375 | 625 | 0.041 | 4.16 | 31.8 | 0.01 | 0.004 |
| | 13 - Pull | | | | | | | | |
| 0.2 | MERV | 36 | 375 | 1041 | 0.061 | 1.52 | 7.5 | 0.22 | 0.1408 |
| | 11 - Pull | | | | | | | | |
| 0.2 | Separate | 99.97 | 375 | 375 | 0.027 | 24.3 | 505.2 | N/A | N/A |
| | HEPA - | | | | | | | | |
| | N/A | | | | | | | | |

As shown in Table 1, HEPA filters have a filtration efficiency of 99.97% that is higher than MERV13 having a filtration efficiency of only 60%, and much higher than MERV11 having a filtration efficiency of only 36%. At a given CADR value, higher filtration efficiency corresponds to lower particle penetration through the filter and lower volumetric flow; lower filtration efficiency corresponds to higher particle penetration through the filter and higher volumetric flow. Average Room Air Velocity was determined using the CFD simulation. Of the filters listed in Table 1, MERV11 represents the filter configuration that permits the most amount of particles and highest volumetric flow through the filter.

The computational fluid dynamics model was also used to model air flow to understand localized differences in velocity. See, Table 2.

TABLE 2

| | | | | | Average | Average |
|---|---|---|---|---|---|---|
| | | | | Room | Velocity at | Velocity in |
| | | | | Average | Example | Region of |
| | Filter Type | | | Velocity | Uninfected | Higher |
| UV | and Air | Filtration | | (m/s) | Occupant | Fluence |
| Source | Stream | Efficiency | CADR | (Numerical | Location | rate |
| (μW/cm²) | Direction | (%) | (m³/hr) | Average) | (m/s) | (m/s) |
| 0.2 | None | N/A | N/A | 0.009 | 0.012 | 0.005 |
| 0.2 | HEPA - Pull | 99.97 | 375 | 0.040 | 0.014 | 0.150 |
| 0.2 | MERV 13- | 60 | 375 | 0.077 | 0.031 | 0.294 |
| | Pull | | | | | |
| 0.2 | MERV 11 - | 36 | 375 | 0.116 | 0.061 | 0.437 |
| | Pull | | | | | |
| 0.2 | Separate | 99.97 | 375 | 0.026 | 0.038 | 0.007 |
| | HEPA - N/A | | | | | |
| 0.2 | HEPA - Push | 99.97 | 375 | 0.070 | 0.073 | 0.190 |
| 0.2 | MERV 13 - | 60 | 375 | 0.087 | 0.049 | 0.386 |
| | Push | | | | | |
| 0.2 | MERV 11 - | 36 | 375 | 0.121 | 0.050 | 0.580 |
| | Push | | | | | |

FIG. 14 provides an air velocity contour plot for a modeled comparative example of a space treated based using a far-UV radiation source alone, without use of an air stream or filter. Table 1 provides data based on particle movement as determined by computation fluid dynamics and Table 2 provides data based on air flow velocity. In the case of the UV source without an air stream or filter, the model calculates minimal air movement in the room resulting in slow, but chaotic, movement of particles throughout the space. While modeling showed variations in air movement in the space, particularly around objects and due to air flow resulting from breath, after normalization of the visualization scale the result shows an effectively still environment. Travel distance and travel time of emitted infectious particles are both high. This dynamic has several effects: if particles were emitted outside the irradiation pattern they may remain outside of it for relatively longer periods, particles that are within the area of irradiation may remain inside of it for relatively longer periods, and the direction of particle movement is more unpredictable. Velocity measurement showed low room average velocity and low average velocity in the region of highest irradiance adjacent to the far-UV radiation source. Minimal, albeit somewhat higher, velocity was observed near the breathing area of the non-infected second occupant.

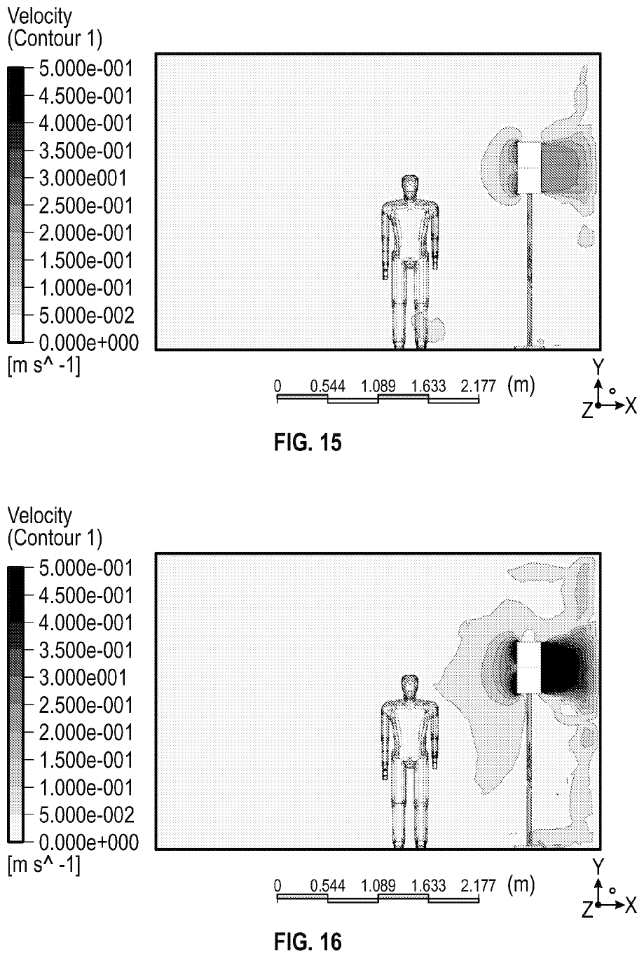

FIG. 15 provides an air velocity contour plot for a modeled example of an apparatus having an integrated far-UV radiation source and air stream unit with HEPA filter performance, which was configured to provide an air stream in the area of irradiated air in a pull configuration that captures irradiated air. In the case of the HEPA filter configured in a pull orientation, particle modeling shows that some spiral motion or lingering of particles is seen as the particles move through the irradiated air space and curve relatively slowly toward the air intake of the apparatus. In this arrangement, the particles are heavily irradiated as they pass through the UV broad cast pattern; and the particles mostly move toward the apparatus without entering the immediate air space of the second, uninfected modeled occupant located facing the infected occupant across the area of irradiation. In this example model, the speed and path of the infectious particles is such that they become extensively inactivated by far-UV radiation before their 1^{st} pass encounter with filter. The far-UV radiation is extremely effective in this configuration and the model calculates a survival fraction of 0.0001, equivalent to a 9 log_e disinfection. Moreover, after the small amount of surviving particles are passed through the HEPA filter, remaining survival fraction is calculated to be further reduced to 3E-08 after a single pass, which is equivalent to a 17 log_e disinfection. Relative to the system lacking an air stream unit, the system configured with an air stream exhibited less chaotic particle movement relative to the system lacking an air stream unit, which can reflect a configuration that responds more predictably in a variety of environments, or in environments where the location of a particle source (e.g., an infected occupant) is not known in advance. Velocity measurement showed relatively low room average velocity, low average velocity in the region of highest irradiance adjacent to the far-UV radiation source, and low air movement at the breathing area of the non-infected second occupant.

FIG. 16 provides an air velocity contour plot for a modeled example of an apparatus having an integrated far-UV radiation source and air stream unit with MERV13 filter performance, which was configured to provide an air stream in the area of irradiated air in a pull configuration that captures irradiated air. In the case of the MERV13 filter configured in a pull orientation, particle modeling showed that less spiral motion of particles is seen relative to the HEPA example as the particles move more directly through the irradiated air space and toward the air intake of the apparatus, and also more noticeably do not enter the immediate air space of the second, uninfected modeled occupant. In this arrangement, the particles are less heavily irradiated as they pass more quickly and directly through the UV broad cast pattern toward the apparatus, yet the particles are still significantly inactivated. The model calculates a survival fraction of 0.01 of the infectious particles before their 1^{st} pass encounter with filter, which represents a 4.5 log_e disinfection. After subsequent passage through the MERV13 filter, the remaining survival fraction is calculated to be further reduced to 0.004 after a single pass, which represents a 5.5 log_e disinfection. Velocity measurement showed moderate room average velocity, moderate velocity in the region of highest irradiance adjacent to the far-UV radiation source, and low air movement at the breathing area of the non-infected second occupant.

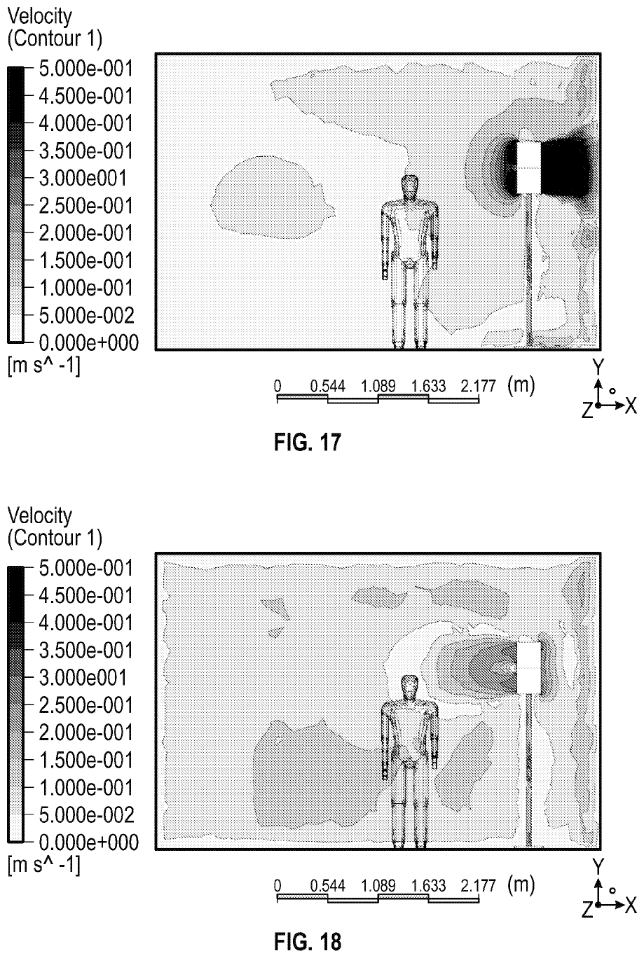

FIG. 17 provides an air velocity contour plot for a modeled example of an apparatus having an integrated far-UV radiation source and air stream unit with MERV11 filter performance, which was configured to provide an air stream in the area of irradiated air in a pull configuration that captures irradiated air. In the case of the MERV11 filter configured in a pull orientation, relative to the MERV13 and HEPA examples, the particle modeling showed that the particles move in an even more direct path through the irradiated air space and toward the air intake of the apparatus, and more significantly avoiding the immediate air space of the second occupant. In this arrangement, the particles are least heavily irradiated as they pass most quickly and directly through the UV broad cast pattern toward the apparatus, yet many particles are still inactivated. The model calculates a survival fraction of 0.22 of the infectious particles before their 1^{st} pass encounter with filter, which represents a 1.5 log_e disinfection. After subsequent passage through the MERV11 filter, the remaining survival fraction is calculated to be further reduced to 0.1408 after a single pass, which represents a 2 log_e disinfection. Particles showed higher velocities in the regions of higher irradiance close to the far-UV apparatus and this effect extended further into the irradiated space relative to the MERV13 and HEPA test examples. Velocity measurement showed relatively high room average velocity, relatively high average velocity in the region of highest irradiance adjacent to the far-UV radiation source, and relatively high average velocity in the breathing area of the non-infected second occupant.

FIG. 18 provides an air velocity contour plot for a modeled example of an apparatus having an integrated far-UV radiation source and air stream unit with HEPA filter performance, which was configured to provide an air stream in the area of irradiated air in a push configuration that exhausts purified air to the area of irradiation. In this configuration, particle modeling showed that the infectious particles were slowly pushed away from the area of the highest intensity irradiation as if pushed away by the purified air, ultimately reaching a boundary of the space, primarily the wall on the opposite side of the space as the far-UV radiation source, before circling back through the room by tracking along the walls, ceiling, and floor to the air intake with occasional vortexes in corner spaces. A higher level of chaotic movement was exhibited in this example. Velocity measurement showed that particles showed higher velocities in the regions of higher irradiance close to the far-UV apparatus. The area of higher velocity extended further into the irradiated space relative to the pull-type configurations. Velocity measurement showed higher room average velocity, higher average velocity in the region of highest irradiance adjacent to the far-UV radiation source, and higher average velocity in the breathing area of the non-infected second occupant for push-configuration relative to pull-configuration HEPA performance values in the model space.

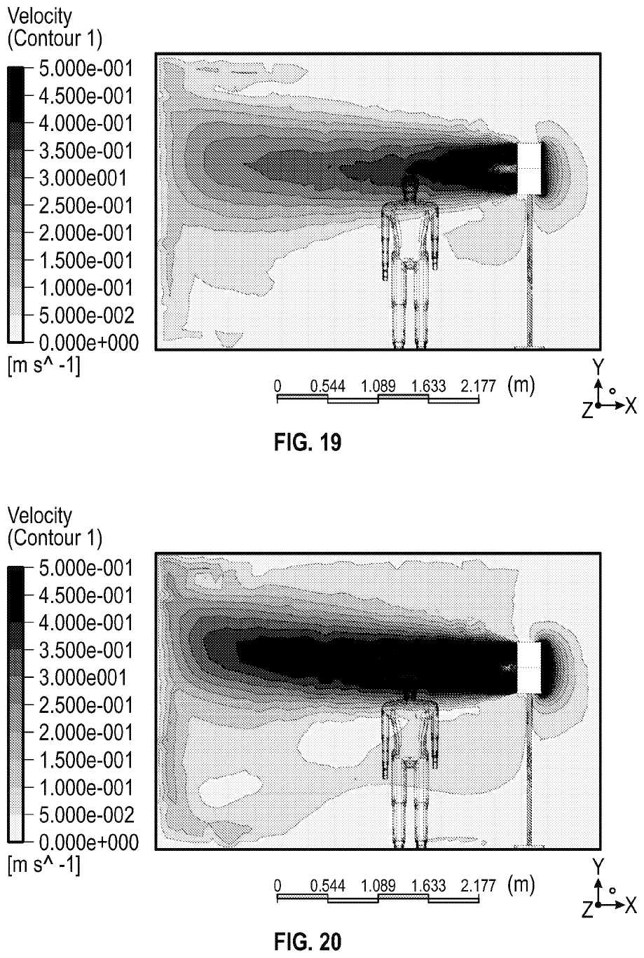

FIG. 19 provides an air velocity contour plot for a modeled example of an apparatus having an integrated far-UV radiation source and air stream unit with MERV13 filter performance, which was configured to provide an air stream in the area of irradiated air in a push configuration that exhausts purified air to the area of irradiation. In this configuration, particle modeling showed that the infectious particles were more pushed away from the area of the higher intensity irradiation as if pushed away by the purified air, ultimately reaching a boundary of the space, primarily the wall on the opposite side of the space as the far-UV radiation source, before circling back through the room. Relative to the HEPA example, particle paths more directly traversed the room space to the air intake with less lingering, and more particle paths passed back through the area of higher intensity far-UV irradiation en route to the air intake. This example configuration also showed a chaotic particle movement paths as particles looped around the room to air intake, but relatively less compared with the example with HEPA performance. Velocity measurement showed that particles showed higher velocities in the regions of higher irradiance close to the far-UV apparatus. The area of higher velocity extended further into the irradiated space relative to the HEPA push-type example and localized velocities were higher. Velocity measurement showed higher room average velocity, higher average velocity in the region of highest irradiance adjacent to the far-UV radiation source, and higher average velocity in the breathing area of the non-infected second occupant for the MERV13 push-configuration example relative to the HEPA push-configuration example in the model space.

FIG. 20 provides an air velocity contour plot for a modeled example of an apparatus having an integrated far-UV radiation source and air stream unit with MERV11 filter performance, which was configured to provide an air stream in the area of irradiated air in a push configuration that exhausts purified air to the area of irradiation. In this configuration, particle modeling showed that the infectious particles were pushed away from the area of the highest intensity irradiation as if pushed away by the purified air, before quickly and more directly back around to the device air intake, often by tracking along a wall or other surface. Particle movement largely avoided the second uninfected occupant. Relative to the HEPA and MERV13 example, the particle paths were more predictable and uniform, and movement of emitted infectious particles shows paths that efficiently moved to apparatus air intake, despite the particle emissions being directed into the exhausted air stream. Thus, this “push” type configuration can exhibit an air stream that advantageously moves particles out of the area of far-UV irradiation and to an air intake of the device. Such air stream may also have secondary advantages with respect to avoiding short-range transmission and facilitating multi-pass purification of contaminants. This example configuration also showed less chaotic particle movement relative to the push-type example with HEPA and MERV13 performance, which can reflect a configuration that responds more predictably in a variety of environments, or in environments where the location of a particle source (e.g., an infected occupant) is not known in advance. Velocity measurement showed that particles showed higher velocities in the regions of higher irradiance close to the far-UV apparatus. The area of higher velocity extended further into the irradiated space relative to the HEPA push-type example and localized velocities were higher. Velocity measurement showed higher room average velocity, higher average velocity in the region of highest irradiance adjacent to the far-UV radiation source, and higher average velocity in the breathing area of the non-infected second occupant for the MERV13 push-configuration example relative to the MERV11 and HEPA push-configuration example in the model space.

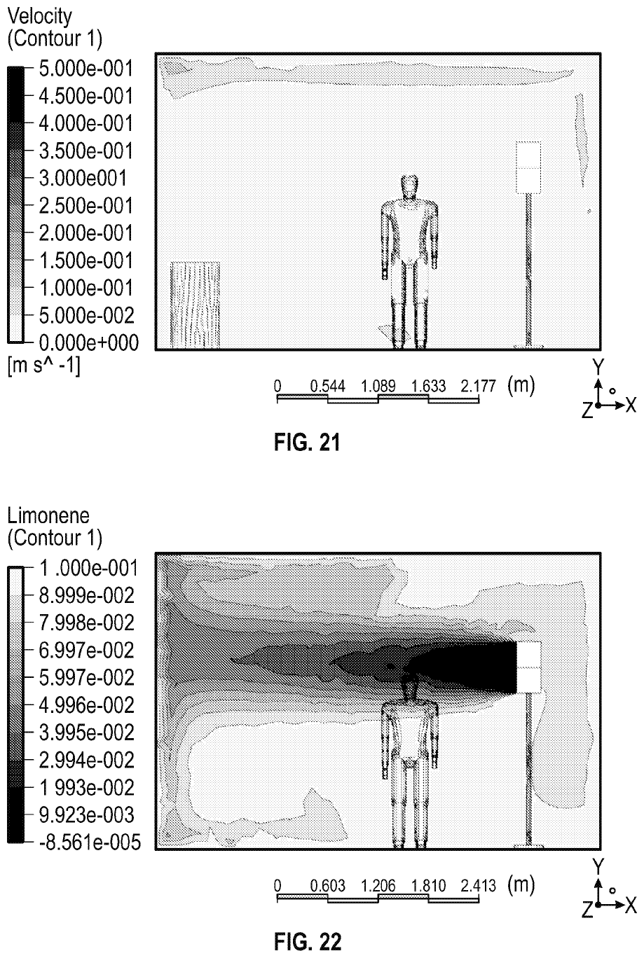

FIG. 21 provides an air velocity contour plot for a modeled comparative example where the far-UV radiation source is separate from a discrete air stream source with HEPA filter performance. The separate air stream source was placed on the floor near a corner, rather than at head height, to approximate the location of air stream that would be generated by portable air purifiers. In this case, particles moved from the particle emitter toward the air stream source, which was located away from the far-UV radiation source. Particles moved relatively slowly and particle paths concentrated in a large loop along the adjacent surfaces of the air stream source largely omitting the area of high fluence rate. Velocity contour also showed faster moving particles near the periphery of the space in this arrangement. Velocity measurement did not indicate higher velocities in the regions of higher irradiance close to the far-UV apparatus or near occupants.

FIG. 22 provides a concentration contour plot illustrating concentration of a VOC in a human-use space treated with an apparatus having a far-UV radiation source and air stream unit with a scrubber, which provides an air stream in the area of irradiated air in a push configuration that exhausts purified air to the area of irradiation. A model was configured where the example far-UV apparatus was configured to have an air stream having a scrubber that removes VOCs and ozone with a 99% capture rate. To simulate an indoor cleaning event, the floor and wall surfaces were modeled to have a limonene concentration of 10,000 ppm, to simulate the space after having been cleaned. The model was run until residuals reached less than 1E-4, and then terminated. In this example, limonene was utilized as a representative VOC or non-pathogenic contaminant. The contour plot shows the distribution of the VOC throughout the space and that a region of lower concentration of limonene is in the region of highest fluence rate in front of the far-UV radiation source. In this case, it appears that purified air displaces limonene out of the high fluence rate irradiated region and circulates it through the room to ultimately reach the apparatus air intake for purification. Regions of lower VOC concentration are evident in the higher fluence rate regions of the far-UV broadcast but also extend to some extent as far as the opposite wall. Here, the air stream provides purified air that pushes VOC and other contaminants away from the high fluence rate region in the front of the far-UV radiation source, and ultimately facilitates movement of the particles through the space for eventual capture and purification. This behavior is beneficial in that it can result in reduced formation of secondary products. The amount of time that contaminants linger in high fluence rate regions corresponds to the amount of secondary product formation. The contaminants can include “non-pathogenic contaminants” such as ozone, oxygenated particles, oxygenated organic small molecules, oxygenated organic macromolecules and materials, oxygenated dust particles, non-pathogenic degradants of pathogens, aggregated particles containing the same, or combinations thereof. For example, VOC and ozone lingering in high fluence rate regions can be generated oxygenated organic small molecules and aggregations of oxygenated particles. Reducing the amount of time such contaminants occupy the high fluence rate regions can significantly reduce the formation of resulting secondary contaminants. Results similar to the limonene concentration map can be observed with other VOCs, ozone, and other contaminants when similarly modeled.

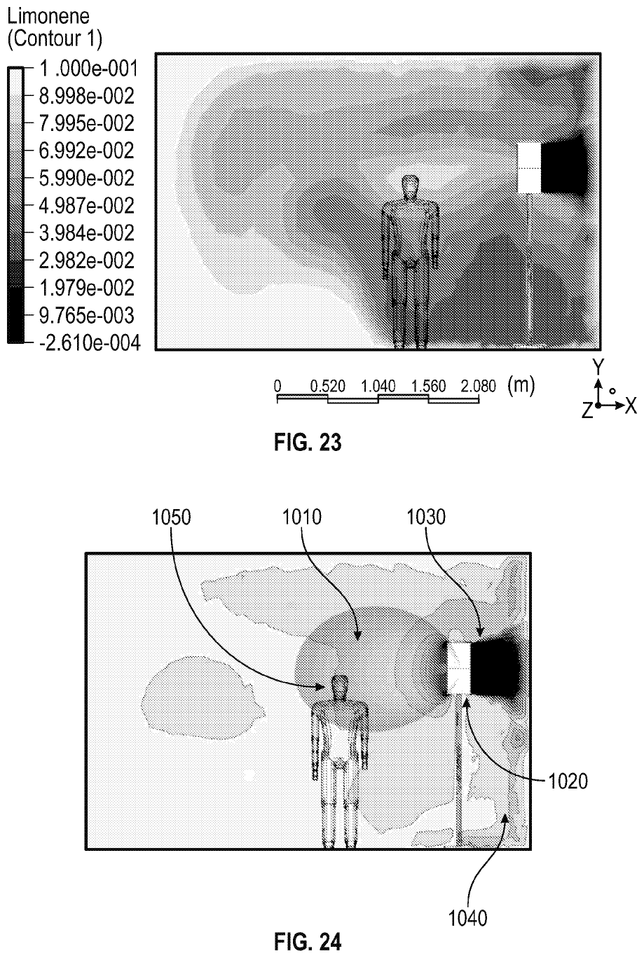

FIG. 23 provides a concentration contour plot illustrating concentration of a VOC in a human-use space treated with an apparatus having a far-UV radiation source and air stream unit, which provides an air stream in the area of irradiated air in a pull configuration that captures irradiated air. The same conditions in this example were used as in the push-type example that assessed VOC concentration. The contour plot shows the distribution of the VOC throughout the space and that a region of lowest concentration of limonene is in exhaust air of the apparatus. The region of high fluence rate in front of the far-UV radiation source shows moderate concentrations of the VOC. Here, the air stream pulls VOC and other contaminants toward the high fluence rate region in front of the far-UV radiation source, captures the contaminants for passage through the apparatus, and then exhausts purified air that circulates around them room. In this configuration, higher relative concentration of VOC in the region of higher irradiation can benefit from faster velocity of particles in the same region. Reduced time of VOC in the irradiated region can result in reduced formation of harmful secondary products. Further, a pull configuration can capture resulting secondary contaminants, which can form in a localized manner in the region of high fluence rate, and capture of such contaminants is advantageous.

In both the pull and push configurations, resulting secondary products can remain low, even as low as below detectable or quantifiable limits, due to the disruption in their formation, subsequent capture, or both.

FIG. 24 provides an air velocity contour plot with a fluence rate contour plot overlay, and illustrates how a far-UV radiation source can be integrated with an air stream unit to position a localized air stream in the areas of high radiation intensity. Region 1010 illustrates a high fluence rate region in front of modeled far-UV apparatus 1020. The region 1010 shows increasing fluence rate closest to the far-UV apparatus 1020 in an inverse square relationship. The apparatus 1020 generates an air stream having high velocity regions aligned with higher fluence rate in region 1010. In the illustrated example, the air stream is provided in a pull-type configuration. The apparatus 1020 captures contaminants, including pathogenic contaminants, pollutants, non-pathogenic contaminants, and/or non-pollutant contaminants that are precursors for undesirable contaminants. Contaminants in the air stream are removed via a scrubber, filter, or both, and purified air 1030 is exhausted from the apparatus. In the illustrated configuration, the purified air 1030 is exhausted as fast moving clean air toward an outer boundary of the space, which facilitates recirculation 1040 of the purified air 1030 back into the inner portion of the space, where one or more occupants 1050 may be located. Occupants can potential be direct or indirect sources of contaminants, but they are also potentially vulnerable to the same. The region of higher UV fluence rate is highly germicidal, yet with higher UV fluence rate the same region is susceptible to higher ozone generation, higher ozone concentration, higher secondary contaminant generation, and higher secondary contaminant concentration. Oxygen in air with UV light can generate ozone, and ozone with VOC can generate secondary oxygenated aerosols—this represents one source of secondary contaminants that can be problematic for far-UV use. The illustrated configuration advantageously provides a solution by providing a localized air stream with higher velocities in one or more region of high fluence rate to disrupt generation of contaminants, capture them for purification, or both. The exhausted purified air 1030 provides clean air and air flow for continued dilution, displacement, and purification of air in the space. Alternative configurations that provide an air stream aligned with areas of high fluence rate are also beneficial. For example, the air stream can be configured in a push configuration where purified air 1030 can be instead pushed into the higher fluence rate region 1010 thus diluting ozone and VOC, and disrupting secondary organic aerosol formation.

Fluid dynamic mixing and localized flow events can be highly complex and their interplay with germicidal UV treatment has not been sufficiently appreciated to date. Certain system configurations can exhibit difficult to predict fluid dynamic behavior in which particle paths can vary greatly based on system state, such as described in the example case of use of a far-UV radiation source without an air stream unit or the example utilizing a separate corner-located air stream unit. When such systems are intended for use in human-use environments, the wide variety of possible human circumstances can lead to undesired outcomes or performance. For example, a given human-use space may have undesirable particles sources arising due to cooking, cleaning, manufacturing, agricultural activities, painting, building, burning, smoking, odor generating activities, or food activities, and yet such spaces may lack control over where or when that particle generation occurs. Positioning a given device in a position of particle generation is not always possible—the case of an unknowingly infected occupant is a clear example. Use of a far-UV radiation apparatus that provides a robust air stream can be provide both localized and/or generalized air flow streams each of which in various examples can have the advantage of improving general performance or minimizing undesired effects such as byproduct formation that can occur in a variety of system states. To date, fluid dynamic mixing, localized flow effects, and their various variables have not been sufficiently appreciated in terms of how they impact desirable performance of far-UV systems and the field has not identified which localized mixing parameters are impactful, effective, or desirable for designing or configuring a device that inactivates pathogens without generating harmful secondary products. Filtration efficiency is one example: a HEPA filter can have a particle removal efficiency greater than 99.97% for airborne particles that are 0.3 microns in diameter or larger, and this is widely considered a desirable aspect for air purification. However, the reduced airflow arising from use of HEPA-type filters can result in a greater influence of certain localized effects—a HEPA filter ideally positioned adjacent to a localized source of undesirable particles can be highly effective for eliminating such particles, but other locations for the HEPA filter can be significantly less effective. In the case of undesirable byproduct formation, undesirable particles can arise from a localized area of UV radiation intensity within the broadcast pattern of a far-UV radiation source. Surprisingly, use of a HEPA filter even within the irradiated space does not in itself sufficiently address the problem of byproduct formation due to low air velocity and particle linger. A desirable solution can be achieved by appropriate orientation of an air stream unit in close proximity to the far-UV source but outside of the UV broadcast pattern so as to provide a robust localized air stream through the adjacent areas of highest UV intensity. Unintuitively, the air stream unit can further benefit from use of a filter system having lower filtration efficiency (i.e., higher penetration), particularly in the case of portable units or other units where fan size, power, or number can be limited, electrical power is limited, or fan noise can impact occupant use and enjoyment of a treated space. As another example, airborne pathogens, non-pathogenic airborne contaminants, non-pollutant contaminants that are precursors for undesirable contaminants, and secondary contaminant byproducts are not always well mixed in treated spaces, and each can be generated and distributed throughout human-use spaces in localized, poorly mixed ways.

The irradiation provided by a far-UV radiation source can be described in terms of a given broadcast pattern where, based on an inverse square relationship, the most intense light in the broadcast pattern is closest to the device. Intensity decays with distance from the source. Undesirable airborne byproducts such as ozone, oxygenated particles, oxygenated organic small molecules, oxygenated organic macromolecules and materials, oxygenated dust particles, non-pathogenic degradants of pathogens, aggregated particles containing the same, or combinations thereof, can arise due to the action of UV light on various precursors, including oxygen, ozone, VOCs, and particle matter. The action of UV light, however, is also what imparts the disinfecting effect of germicidal UV light. Various presently described apparatus, systems, and methods provide a solution to the problem of undesirable airborne byproducts by being configured to provide increased velocity of particles and gases at proximate regions of high UV radiation thus reducing irradiation time during first pass before facilitating conveyance of the contaminant to a filter or scrubber system for elimination, or subsequent recirculation for additional UV irradiation. UV-based generation of harmful byproducts is a potential safety concern for far-UV based technology. Various far-UV apparatus, systems, and methods described herein provide a solution by utilizing localized air streams at regions of high UV radiation intensity.

The above description is intended to be illustrative, and not restrictive. For example, each of the above-described aspects and features should be understood to optionally be used in any combination with the others. Other embodiments can be used, such as by one of ordinary skill in the art upon reviewing the above description. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### Exemplified Aspects

The following aspects are provided as example aspects of the various disclosed subject matter:

Aspect 1 provides an apparatus for inactivating a pathogen in air, comprising:
an air stream unit comprising an air stream housing defining an air flow chamber, an air intake, and an air exhaust;
a filter system comprising a mechanical filter, a scrubber, or both, disposed in the air flow chamber;
a fan system configured to provide an air stream through the air intake, air flow chamber, and air exhaust; and
a far-UV radiation source that emits radiation comprising one or more wavelengths of about 210 nm to about 230 nm, wherein the far-UV radiation source is integrated with the air stream unit external to the air flow chamber and adjacent to at least one of the air intake or air exhaust, and the far-UV radiation source is positioned to direct radiation away from the air stream unit, the air stream housing, the filter if present, the scrubber if present, or a combination thereof.

Aspect 2 provides an apparatus of Aspect 1, wherein the air stream housing and filter system is substantially free of direct radiation from the far-UV radiation source.

Aspect 3 provides an apparatus of Aspect 1 or 2, wherein the air stream housing is configured to displace irradiated air proximate to the far-UV radiation source.

Aspect 4 provides an apparatus of any one of Aspects 1-3, wherein the air intake is adjacent to the far-UV radiation source and the air stream housing is configured to capture irradiated air proximate to the far-UV radiation source.

Aspect 5 provides an apparatus of any one of Aspects 1-4, wherein the air exhaust is directed away from the far-UV radiation source and optionally directed to a ceiling, wall, floor, or deflector to circulate air in an occupiable space.

Aspect 6 provides an apparatus of any one of Aspects 1-5, wherein the air intake is adjacent to the far-UV radiation source and the air stream housing is configured to exhaust the air stream away from irradiated air proximate to the far-UV radiation source.

Aspect 7 provides an apparatus of any one of Aspects 1-6, wherein the air exhaust is adjacent to the far-UV radiation source and the air stream housing is configured to exhaust the air stream into a space irradiated by the far-UV radiation source.

Aspect 8 provides an apparatus of any one of Aspects 1-7, wherein the air stream housing is configured to displace irradiated air in a direction substantially parallel to a direction of the emitted radiation.

Aspect 9 provides an apparatus of any one of Aspects 1-8, wherein the air stream housing is configured to capture irradiated air and displace it in a direction substantially opposite to a direction of the emitted radiation.

Aspect 10 provides an apparatus of any one of Aspects 1-9, wherein the air stream housing is configured to displace irradiated air in a direction substantially perpendicular to a direction of the emitted radiation.

Aspect 11 provides an apparatus of any one of Aspects 1-10, wherein the air stream housing comprises the mechanical filter.

Aspect 12 provides an apparatus of any one of Aspects 1-11, wherein the one or more mechanical filters have a penetration of 5% or greater at 0.3 microns.

Aspect 13 provides an apparatus of any one of Aspects 1-12, wherein the one or more mechanical filters have mechanical filters have a penetration of 40% or greater at 0.3 microns.

Aspect 14 provides an apparatus of any one of Aspects 1-13, wherein the mechanical filter is not a HEPA filter, does not meet a HEPA standard, does not have a filter efficiency of greater than 99%, or any combination thereof.

Aspect 15 provides an apparatus of any one of Aspects 1-14, wherein the mechanical filter is not a MERV16 or greater filter per MERV Standard 52.2.

Aspect 16 provides an apparatus of any one of Aspects 1-15, wherein the mechanical filter is not suitable as a single pass filter for achieving an ISO 20E or better using ISO 29463-1 2017.

Aspect 17 provides an apparatus of any one of Aspects 1-16, wherein the mechanical filter has a single pass efficiency of less than 80%.

Aspect 18 provides an apparatus of any one of Aspects 1-17, wherein the mechanical filter has a flow rate through the filter of at least 50 CFM at a pressure drop of 0.25 or less.

Aspect 19 provides an apparatus of any one of Aspects 1-18, wherein the mechanical filter is not borosilicate microfibers.

Aspect 20 provides an apparatus of any one of Aspects 1-19, wherein the mechanical filter is a pleated filter having a surface area of 20 m² to 80 m².

Aspect 21 provides an apparatus of any one of Aspects 1-20, wherein the mechanical filter has a single pass efficiency of ISO 15 E or less and fan pressure drop curve up to 0.1 at 100 CFM to up to 0.5 at 500 CFM.

Aspect 22 provides an apparatus of any one of Aspects 1-21, wherein the filter system consists of two MERV13 filters.

Aspect 23 provides an apparatus of any one of Aspects 1-22, wherein the air flow chamber comprises the scrubber.

Aspect 24 provides an apparatus of any one of Aspects 1-23, wherein the scrubber an active scrubber.

Aspect 25 provides an apparatus of any one of Aspects 1-24, wherein the scrubber an active scrubber that is powered by a power supply shared with the far-UV radiation source and the fan system.

Aspect 26 provides an apparatus of any one of Aspects 1-25, wherein the scrubber is a passive scrubber.

Aspect 27 provides an apparatus of any one of Aspects 1-26, wherein the scrubber is zeolite.

Aspect 28 provides an apparatus of any one of Aspects 1-27, wherein the scrubber is activated charcoal.

Aspect 29 provides an apparatus of any one of Aspects 1-28, wherein the fan system is one or more tubeaxial fans.

Aspect 30 provides an apparatus of any one of Aspects 1-29, wherein the fan system is one or more fans each having a diameter of 500 mm or less.

Aspect 31 provides an apparatus of any one of Aspects 1-30, wherein the fan system is one or more fans each about 6 W or less, and taken together the fan system is 200 W or less.

Aspect 32 provides an apparatus of any one of Aspects 1-31, wherein the fan system is one or more tubeaxial fans each providing a static pressure of about 0.5 inches H₂O or less, and taken together the fan system provides a static pressure of about 0.5 inches H₂O or less.

Aspect 33 provides an apparatus of any one of Aspects 1-32, wherein the fan system provides a static pressure against filter of about 0.1 inches H₂O or less at a CFM of at least 100 to about 0.5 inches H₂O or less at a CFM of at least 500.

Aspect 34 provides an apparatus of any one of Aspects 1-33, wherein the fan system is two to six tubeaxial fans having a diameter of 150 mm or less.

Aspect 35 provides an apparatus of any one of Aspects 1-34, wherein the far-UV radiation source is a barrier discharge lamp.

Aspect 36 provides an apparatus of any one of Aspects 1-35, wherein the far-UV radiation source is a KrCl excimer lamp.

Aspect 37 provides an apparatus of any one of Aspects 1-36, wherein the far-UV radiation source comprises a bulb having an annular body having an outer surface and defining an internal discharge cavity, wherein the annular body has a major axial dimension and a minor radial dimension, and comprises an electrode in the internal discharge cavity that traverses along the major axial dimension of the annular body.

Aspect 38 provides an apparatus of any one of Aspects 1-37, wherein the far-UV radiation source comprises a lamp housing, a bulb, a reflector, and a window.

Aspect 39 provides an apparatus of any one of Aspects 1-38, wherein the far-UV radiation source comprises an optical filter.

Aspect 40 provides an apparatus of any one of Aspects 1-39, wherein the far-UV radiation source comprises an optical filter that substantially prevents transmittance of one or more wavelength outside of the range between about 200 nm to about 230 nm.

Aspect 41 provides an apparatus of any one of Aspects 1-40, wherein the far-UV radiation source comprises an optical filter that substantially transmits one or more wavelength outside of the range between about 200 nm to about 230 nm.

Aspect 42 provides an apparatus of any one of Aspects 1-41, wherein the far-UV radiation source is integrated with the air stream housing via a fastener, an adhesive, an interlocking part, welding, or being molded together with an exterior surface of the air stream housing.

Aspect 43 provides an apparatus of any one of Aspects 1-42, wherein the far-UV radiation source is integrated with the air stream housing by placement at an air intake or air exhaust of the air stream housing.

Aspect 44 provides an apparatus of any one of Aspects 1-43, wherein the far-UV radiation source is integrated with the air stream housing via a shared stand or mount.

Aspect 45 provides an apparatus of any one of Aspects 1-44, configured as a portable system.

Aspect 46 provides an apparatus of any one of Aspects 1-45, further comprising a floor stand, tabletop stand, wall mount, or ceiling mount.

Aspect 47 provides an apparatus of any one of Aspects 1-46, further comprising a telescoping or collapsible stand.

Aspect 48 provides an apparatus of any one of Aspects 1-47, which is 60 lbs, or less.

Aspect 49 provides an apparatus of any one of Aspects 1-48, which has a volume of 20 sq ft or less.

Aspect 50 provides an apparatus of any one of Aspects 1-49, which has a height, width, and length, if present, of 40 inches or less.

Aspect 51 provides an apparatus of any one of Aspects 1-50, which has a base of 4 sq ft or less.

Aspect 52 provides an apparatus of any one of Aspects 1-51, which has a base less than 1-5% of the space for which it is configured to treat.

Aspect 53 provides an apparatus of any one of Aspects 1-52, which provides an apparatus comprises a stand configurable to a height of at least 4 feet.

Aspect 54 provides an apparatus of any one of Aspects 1-53, which provides an apparatus comprises one or more handles.

Aspect 55 provides an apparatus of any one of Aspects 1-54, configured for use as a permanent fixture.

Aspect 56 provides an apparatus of any one of Aspects 1-55, which is configured for attaching the air stream housing to a return or a supply of an HVAC system.

Aspect 57 provides an apparatus of any one of Aspects 1-56, wherein the far-UV radiation source is integrated with the air stream housing via a shared power supply.

Aspect 58 provides an apparatus of any one of Aspects 1-57, wherein the far-UV radiation source is located within 24 inches of the air intake or air exhaust.

Aspect 59 provides an apparatus of any one of Aspects 1-58, wherein the air intake and air exhaust provide one ore more rotary vortices in a room the apparatus is positioned.

Aspect 60 provides an apparatus of any one of Aspects 1-59, wherein a dimension of the air stream housing, air intake, or air exhaust is approximately equal to a dimension of the far-UV radiation source or a bulb of the far-UV radiation source.

Aspect 61 provides an apparatus of any one of Aspects 1-60, wherein the air stream housing is positioned to approximate an edge of the broadcast pattern of the far-UV radiation source.

Aspect 62 provides an apparatus of any one of Aspects 1-61, wherein the air stream housing, fan system, filter, and scrubber, if present, provide an air stream at the air intake and the air exhaust of at least 100 ft³/min.

Aspect 63 provides an apparatus of any one of Aspects 1-62, further comprising an external reflector or diffusor.

Aspect 64 provides an apparatus of any one of Aspects 1-63, further comprising a handle, a cap, or a hinged cover.

Aspect 65 provides an apparatus of any one of Aspects 1-64, further comprising a sensor that collects spectrophotometric data, irradiance data, or spectral data of one or more far-UV radiation sources, air quality data, gas content data, sound data, light data, usage data, system status, maintenance-related data, room context data, room occupancy data, or any combination thereof.

Aspect 66 provides an apparatus of any one of Aspects 1-65, further comprising a control system that receives sensor data and adjusts a performance parameter of the apparatus.

Aspect 67 provides an apparatus of any one of Aspects 1-66, further comprising a control system that receives sensor data and adjusts one or more performance parameter of one or more of the air stream unit, the filter system, the mechanical filter, the scrubber, the fan, and the far-UV radiation source.

Aspect 68 provides an apparatus of any one of Aspects 1-67, further comprising a reporting system that collects sensor data and notifies a user of suggested maintenance or changes to a performance parameter.

Aspect 69 provides an apparatus of any one of Aspects 1-68, wherein the far-UV radiation source is integrated with the air stream housing to provide a cuboid or cylindrical shape.

Aspect 70 provides an apparatus of any one of Aspects 1-69, wherein the air intake or air exhaust that is adjacent to the far-UV radiation source is a shape sharing at least one dimension with a dimension of the far-UV radiation source or a reflector, bulb, or lens of the far-UV radiation source.

Aspect 71 provides an apparatus of any one of Aspects 1-70, wherein the air intake or air exhaust that is adjacent to the far-UV radiation source is substantially rectangular having an edge substantially parallel to an edge of the far-UV radiation source or a reflector, bulb, or lens of the far-UV radiation source.

Aspect 72 provides an apparatus of any one of Aspects 1-71, wherein the air intake or air exhaust that is adjacent to the far-UV radiation source is annular and circumscribes the far-UV radiation source.

Aspect 73 provides an apparatus of any one of Aspects 1-72, wherein the air stream housing is configured to provide a diffuse exhaust.

Aspect 74 provides an apparatus of any one of Aspects 1-73, wherein the air exhaust is not suitable for providing an airflow wall.

Aspect 75 provides an apparatus of any one of Aspects 1-74, wherein the air exhaust facilitates scattering of droplets, aerosols, and particles in air.

Aspect 76 provides an apparatus of any one of Aspects 1-75, wherein the far-UV radiation source does not contain a light guide.

Aspect 77 provides an apparatus of any one of Aspects 1-76, wherein a ratio of total fan power/total optical power is about 500-1,000 with an air stream unit configured for a volumetric air flow of at least 500 m³/hr.

Aspect 78 provides an apparatus of any one of Aspects 1-77, wherein a ratio of total optical power/volumetric air flow is about 0.05 mW/(m³/hr) to about 1.00 mW/(m³/hr) with 100 W or less of total fan power, and 150 W or less of total power to the apparatus.

Aspect 79 provides an apparatus of any one of Aspects 1-78, wherein a ratio of total optical power/total filter surface area of about 0.5 mW/m² to about 10 mW/m² with an air stream unit configured for a volumetric air flow of at least 850 m³/hr and a 100 W or less of total fan power.

Aspect 80 provides an apparatus of any one of Aspects 1-79, which provides a clean air delivery rate (CADR) of at least 300 m³/h.

Aspect 81 provides an apparatus of any one of Aspects 1-80, which provides a volumetric air flow of 350 m³/h.

Aspect 82 provides an apparatus of any one of Aspects 1-81, which provides an air velocity of at least 0.6 m/sec 0.25 m from the air exhaust.

Aspect 83 provides a portable kit for inactivating a pathogen in air, comprising an apparatus of any one of Aspects 1-82.

Aspect 84 provides a system for inactivating a pathogen in air, comprising:
a far-UV radiation source configured for irradiating air of a human-use space, wherein the far-UV radiation source emits radiation comprising one or more wavelengths of about 210 nm to about 230 nm;
an air stream unit comprising an air stream housing that defines an air flow chamber, an air intake, and an air exhaust,
a fan system configured to displace irradiated air proximate to the radiation source and
provide an air stream through the air intake, air flow chamber, and air exhaust; optionally, a filter disposed in the air flow chamber configured for removing non-pathogenic particles from the air; and
optionally, a scrubber disposed in the air flow chamber configured for removing reactive agents from the air;
wherein the far-UV radiation source is integrated with the air stream unit external to the air flow chamber and adjacent to at least one of the air intake or air exhaust, and the far-UV radiation source is positioned such that substantially all radiation is directed away from the air stream housing.

Aspect 85 provides a system of Aspect 84 involving or utilizing involving or utilizing the apparatus of any one of Aspects 1-83 or a component or feature thereof.

Aspect 86 provides a system of Aspect 84 or 85, wherein the air stream housing and filter system is substantially free of direct radiation from the far-UV radiation source.

Aspect 87 provides a system of any one of Aspects 84-86, wherein the air stream housing is configured to displace irradiated air proximate to the far-UV radiation source.

Aspect 88 provides a system of any one of Aspects 84-87, wherein the air intake is adjacent to the far-UV radiation source and the air stream housing is configured to capture irradiated air proximate to the far-UV radiation source.

Aspect 89 provides a system of any one of Aspects 84-88, wherein the air exhaust is directed away from the far-UV radiation source and optionally directed to a ceiling, wall, floor, or deflector to circulate air in an occupiable space.

Aspect 90 provides a system of any one of Aspects 84-89, wherein the air intake is adjacent to the far-UV radiation source and the air stream housing is configured to exhaust the air stream away from irradiated air proximate to the far-UV radiation source.

Aspect 91 provides a system of any one of Aspects 84-90, wherein the air exhaust is adjacent to the far-UV radiation source and the air stream housing is configured to exhaust the air stream into a space irradiated by the far-UV radiation source.

Aspect 92 provides a system of any one of Aspects 84-91, wherein the air stream housing is configured to displace irradiated air in a direction substantially parallel to a direction of the emitted radiation.

Aspect 93 provides a system of any one of Aspects 84-92, wherein the air stream housing is configured to capture irradiated air and displace it in a direction substantially opposite to a direction of the emitted radiation.

Aspect 94 provides a system of any one of Aspects 84-93, wherein the air stream housing is configured to displace irradiated air in a direction substantially perpendicular to a direction of the emitted radiation.

Aspect 95 provides a system of any one of Aspects 84-94, wherein the air stream housing comprises a mechanical filter.

Aspect 96 provides a system of any one of Aspects 84-95, wherein the air stream housing comprises one or more mechanical filters have a penetration of 5% or greater at 0.3 microns, individually, taken together, or both.

Aspect 97 provides a system of any one of Aspects 84-96, wherein the air stream housing comprises one or more mechanical filters have mechanical filters have a penetration of 40% or greater at 0.3 microns, individually, taken together, or both.

Aspect 98 provides a system of any one of Aspects 84-97, wherein the air stream housing comprises one or more mechanical filters that are not a HEPA filter, does not meet a HEPA standard, and does not have a filter efficiency of greater than 99%, taken individually, taken together, or both.

Aspect 99 provides a system of any one of Aspects 84-98, wherein the air stream housing comprises one or more mechanical filters that are not a MERV16 or greater filter per MERV Standard 52.2, individually, taken together, or both.

Aspect 100 provides a system of any one of Aspects 84-99, wherein the air stream housing comprises one or more mechanical filters that are not suitable as a single pass filter for achieving an ISO 20E or better using ISO 29463-1 2017, individually, taken together, or both.

Aspect 101 provides a system of any one of Aspects 84-100, wherein the air stream housing comprises one or more mechanical filters that have a single pass efficiency of less than 80%, individually, taken together, or both.

Aspect 102 provides a system of any one of Aspects 84-101, wherein the air stream housing comprises one or more mechanical filters that have a flow rate through the filter of at least 50 CFM at a pressure drop of 0.25 or less, individually, taken together, or both.

Aspect 103 provides a system of any one of Aspects 84-102, wherein the air stream housing comprises one or more mechanical filters that are not borosilicate microfibers.

Aspect 104 provides a system of any one of Aspects 84-103, wherein the air stream housing comprises one or more mechanical filters that have a pleated filter having a surface area of 20 m² to 80 m², individually, taken together, or both.

Aspect 105 provides a system of any one of Aspects 84-104, wherein the air stream housing comprises one or more mechanical filters that have a single pass efficiency of ISO 15 E or less and fan pressure drop curve up to 0.1 at 100 CFM to up to 0.5 at 500 CFM, individually, taken together, or both.

Aspect 106 provides a system of any one of Aspects 84-105, wherein the air stream housing has a filter system consisting of two MERV13 filters.

Aspect 107 provides a system of any one of Aspects 84-106, wherein the air flow chamber comprises a scrubber.

Aspect 108 provides a system of any one of Aspects 84-107, wherein the air flow chamber comprises a active scrubber.

Aspect 109 provides a system of any one of Aspects 84-108, wherein the air flow chamber comprises an active scrubber that is powered by a power supply shared with the far-UV radiation source and the fan system.

Aspect 110 provides a system of any one of Aspects 84-109, wherein the air flow chamber comprises a passive scrubber.

Aspect 111 provides a system of any one of Aspects 84-110, wherein the air flow chamber comprises zeolite.

Aspect 112 provides a system of any one of Aspects 84-111, wherein the air flow chamber comprises activated charcoal.

Aspect 113 provides a system of any one of Aspects 84-112, wherein the fan system is one or more tubeaxial fans.

Aspect 114 provides a system of any one of Aspects 84-113, wherein the fan system is one or more fans each having a diameter of 500 mm or less.

Aspect 115 provides a system of any one of Aspects 84-114, wherein the fan system is one or more fans each about 6 W or less, and taken together the fan system is 200 W or less.

Aspect 116 provides a system of any one of Aspects 84-115, wherein the fan system is one or more tubeaxial fans each providing a static pressure of about 0.5 inches H₂O or less, and taken together the fan system provides a static pressure of about 0.5 inches H₂O or less.

Aspect 117 provides a system of any one of Aspects 84-116, wherein the fan system provides a static pressure against filter of about 0.1 inches H₂O or less at a CFM of at least 100 to about 0.5 inches H₂O or less at a CFM of at least 500.

Aspect 118 provides a system of any one of Aspects 84-117, wherein the fan system is two to six tubeaxial fans having a diameter of 150 mm or less.

Aspect 119 provides a system of any one of Aspects 84-118, wherein the far-UV radiation source is a barrier discharge lamp.

Aspect 120 provides a system of any one of Aspects 84-119, wherein the far-UV radiation source is a KrCl excimer lamp.

Aspect 121 provides a system of any one of Aspects 84-120, wherein the far-UV radiation source comprises a bulb having an annular body having an outer surface and defining an internal discharge cavity, wherein the annular body has a major axial dimension and a minor radial dimension, and comprises an electrode in the internal discharge cavity that traverses along the major axial dimension of the annular body.

Aspect 122 provides a system of any one of Aspects 84-121, wherein the far-UV radiation source comprises a lamp housing, a bulb, a reflector, and a window.

Aspect 123 provides a system of any one of Aspects 84-122, wherein the far-UV radiation source comprises an optical filter.

Aspect 124 provides a system of any one of Aspects 84-123, wherein the far-UV radiation source comprises an optical filter that substantially prevents transmittance of one or more wavelength outside of the range between about 200 nm to about 230 nm.

Aspect 125 provides a system of any one of Aspects 84-124, wherein the far-UV radiation source comprises an optical filter that substantially transmits one or more wavelength outside of the range between about 200 nm to about 230 nm.

Aspect 126 provides a system of any one of Aspects 84-125, wherein the far-UV radiation source is integrated with the air stream housing via a fastener, an adhesive, an interlocking part, welding, or being molded together with an exterior surface of the air stream housing.

Aspect 127 provides a system of any one of Aspects 84-126, wherein the far-UV radiation source is integrated with the air stream housing by placement at an air intake or air exhaust of the air stream housing.

Aspect 128 provides a system of any one of Aspects 84-127, wherein the far-UV radiation source is integrated with the air stream housing via a shared stand or mount.

Aspect 129 provides a system of any one of Aspects 84-128, configured as a portable system.

Aspect 130 provides a system of any one of Aspects 84-129, further comprising a floor stand, tabletop stand, wall mount, or ceiling mount.

Aspect 131 provides a system of any one of Aspects 84-130, further comprising a telescoping or collapsible stand.

Aspect 132 provides a system of any one of Aspects 84-131, wherein the air stream housing is attached to a return or a supply of an HVAC system.

Aspect 133 provides a system of any one of Aspects 84-132, wherein the far-UV radiation source is integrated with the air stream housing via a shared power supply.

Aspect 134 provides a system of any one of Aspects 84-133, wherein the far-UV radiation source is located within 24 inches of the air intake or air exhaust.

Aspect 135 provides a system of any one of Aspects 84-134, wherein the air intake and air exhaust provide one or more rotary vortices in the air.

Aspect 136 provides a system of any one of Aspects 84-135, wherein the air stream housing, fan system, filter, and scrubber, if present, provide an air stream at the air intake and the air exhaust of at least 100 ft³/min.

Aspect 137 provides a system of any one of Aspects 84-136, further comprising an external reflector or diffusor.

Aspect 138 provides a system of any one of Aspects 84-137, further comprising a handle, a cap, or a hinged cover configured for transporting the far-UV radiation source and the air stream unit together.

Aspect 139 provides a system of any one of Aspects 84-138, further comprising a sensor that collects spectrophotometric data, irradiance data, or spectral data of one or more far-UV radiation sources, air quality data, gas content data, sound data, light data, usage data, system status, maintenance-related data, room context data, room occupancy data, or any combination thereof.

Aspect 140 provides a system of any one of Aspects 84-139, further comprising a control system that receives sensor data and adjusts a performance parameter of the system.

Aspect 141 provides a system of any one of Aspects 84-140, further comprising a control system that receives sensor data and adjusts one or more performance parameter of one or more of the air stream unit, the filter system, the mechanical filter, the scrubber, the fan, and the far-UV radiation source.

Aspect 142 provides a system of any one of Aspects 84-141, further comprising a reporting system that collects sensor data and notifies a user of suggested maintenance or changes to a performance parameter.

Aspect 143 provides a system of any one of Aspects 84-142, wherein the air intake or air exhaust that is adjacent to the far-UV radiation source is a shape sharing at least one dimension with a dimension of the far-UV radiation source or a reflector, bulb, or lens of the far-UV radiation source.

Aspect 144 provides a system of any one of Aspects 84-143, wherein the air intake or air exhaust that is adjacent to the far-UV radiation source is substantially rectangular having an edge substantially parallel to an edge of the far-UV radiation source or a reflector, bulb, or lens of the far-UV radiation source.

Aspect 145 provides a system of any one of Aspects 84-144, wherein the air intake or air exhaust that is adjacent to the far-UV radiation source is annular and circumscribes the far-UV radiation source.

Aspect 146 provides a system of any one of Aspects 84-145, wherein the air stream housing is configured to provide a diffuse exhaust.

Aspect 147 provides a system of any one of Aspects 84-146, wherein the air exhaust is not suitable for providing an airflow wall.

Aspect 148 provides a system of any one of Aspects 84-147, wherein the air exhaust facilitates scattering of droplets, aerosols, and particles in air.

Aspect 149 provides a system of any one of Aspects 84-148, wherein the far-UV radiation source does not contain a light guide.

Aspect 150 provides a system of any one of Aspects 84-149, wherein a ratio of total fan power/total optical power is about 500-1,000 with an air stream unit configured for a volumetric air flow of at least 500 m³/hr.

Aspect 151 provides a system of any one of Aspects 84-150, wherein a ratio of total optical power/volumetric air flow is about 0.05 mW/(m³/hr) to about 1.00 mW/(m³/hr) with 100 W or less of total fan power, and 150 W or less of total power to the system.

Aspect 152 provides a system of any one of Aspects 84-151, wherein a ratio of total optical power/total filter surface area of about 0.5 mW/m² to about 10 mW/m² with an air stream unit configured for a volumetric air flow of at least 850 m³/hr and a 100 W or less of total fan power.

Aspect 153 provides a system of any one of Aspects 84-152, which provides a clean air delivery rate (CADR) of at least 300 m³/h.

Aspect 154 provides a system of any one of Aspects 84-153, which provides a volumetric air flow of 350 m³/h.

Aspect 155 provides a system of any one of Aspects 84-154, which provides an air velocity of at least 0.6 m/sec 0.25 m from the air exhaust.

Aspect 156 provides a method for inactivating a pathogen in air, comprising:
irradiating air in a human-use space with radiation from a far-UV radiation source, wherein the radiation is configured to inactivate a pathogen in air and comprises one or more wavelengths of about 210 nm to about 230 nm; generating an air stream through an air stream unit and displacing irradiated air proximate to the far-UV radiation source using a fan system, wherein the air stream unit comprises an air stream housing that defines an air flow chamber, an air intake, and an air exhaust;
optionally, removing non-pathogenic particles from the air stream in the air stream housing; and
optionally, removing reactive agents from the air stream in the air stream housing;
wherein the far-UV radiation source is integrated with the air stream unit external to the air flow chamber and adjacent to at least one of the air intake or air exhaust, and the far-UV radiation source is positioned such that substantially all radiation is directed away from the air stream housing.

Aspect 157 provides the method of Aspect 156 involving or utilizing the apparatus or system of any one of Aspects 1-155 or a component or feature thereof.

Aspect 158 provides a method of Aspect 156 or 157, wherein the stream housing and filter system is substantially free of direct radiation from the far-UV radiation source.

Aspect 159 provides a method of any one of Aspects 156-158, wherein the air stream is oriented to displace irradiated air proximate to the far-UV radiation source.

Aspect 160 provides a method of any one of Aspects 156-159, wherein the air stream captures irradiated air proximate to the far-UV radiation source.

Aspect 161 provides a method of any one of Aspects 156-160, wherein the air exhaust is directed away from the far-UV radiation source and optionally directed to a ceiling, wall, floor, or deflector to circulate air in an occupiable space.

Aspect 162 provides a method of any one of Aspects 156-161, wherein the air intake is adjacent to the far-UV radiation source and the air stream exhausts the air stream away from irradiated air proximate to the far-UV radiation source.

Aspect 163 provides a method of any one of Aspects 156-162, wherein the air exhaust is adjacent to the far-UV radiation source and the air stream exhausts the air stream into a space irradiated by the far-UV radiation source.

Aspect 164 provides a method of any one of Aspects 156-163, wherein the air stream displaces irradiated air in a direction substantially parallel to a direction of the emitted radiation.

Aspect 165 provides a method of any one of Aspects 156-164, wherein the air stream captures irradiated air and displaces it in a direction substantially opposite to a direction of the emitted radiation.

Aspect 166 provides a method of any one of Aspects 156-165, wherein the air stream displaces irradiated air in a direction substantially perpendicular to a direction of the emitted radiation.

Aspect 167 provides a method of any one of Aspects 156-166, wherein the air stream housing comprises a mechanical filter.

Aspect 168 provides a method of any one of Aspects 156-167, wherein the air stream housing comprises one or more mechanical filters that have a penetration of 5% or greater at 0.3 microns, each individually, taken together, or both.

Aspect 169 provides a method of any one of Aspects 156-168, wherein the air stream housing comprises one or more mechanical filters that have a penetration of 40% or greater at 0.3 microns, each individually, taken together, or both.

Aspect 170 provides a method of any one of Aspects 156-169, wherein the air stream housing comprises one or more mechanical filters that are not a HEPA filter, does not meet a HEPA standard, and does not have a filter efficiency of greater than 99%, taken individually, taken together, or both.

Aspect 171 provides a method of any one of Aspects 156-170, wherein the air stream housing comprises one or more mechanical filters that are not a MERV16 or greater filter per MERV Standard 52.2, each individually, taken together, or both.

Aspect 172 provides a method of any one of Aspects 156-171, wherein the air stream housing comprises one or more mechanical filters that are not suitable as a single pass filter for achieving an ISO 20E or better using ISO 29463-1 2017, each individually, taken together, or both.

Aspect 173 provides a method of any one of Aspects 156-172, wherein the air stream housing comprises one or more mechanical filters that have a single pass efficiency of less than 80%, each individually, taken together, or both.

Aspect 174 provides a method of any one of Aspects 156-173, wherein the air stream housing comprises one or more mechanical filters that have a flow rate through the one or more filters of at least 50 CFM at a pressure drop of 0.25 or less, each individually, taken together, or both.

Aspect 175 provides a method of any one of Aspects 156-174, wherein the air stream housing comprises one or more mechanical filters that are not borosilicate microfibers.

Aspect 176 provides a method of any one of Aspects 156-175, wherein the air stream housing comprises one or more mechanical filters that is a pleated filter having a surface area of 20 m² to 80 m², each individually, taken together, or both.

Aspect 177 provides a method of any one of Aspects 156-176, wherein the air stream housing comprises one or more mechanical filters that has a single pass efficiency of ISO 15 E or less and fan pressure drop curve up to 0.1 at 100 CFM to up to 0.5 at 500 CFM, each individually, taken together, or both.

Aspect 178 provides a method of any one of Aspects 156-177, wherein the air stream housing comprises a filter system consisting of two MERV13 filters.

Aspect 179 provides a method of any one of Aspects 156-178, wherein the air stream housing comprises a scrubber.

Aspect 180 provides a method of any one of Aspects 156-179, wherein the air stream housing comprises an active scrubber.

Aspect 181 provides a method of any one of Aspects 156-180, wherein the air stream housing comprises an active scrubber that is powered by a power supply shared with the far-UV radiation source and the fan system.

Aspect 182 provides a method of any one of Aspects 156-181, wherein the air stream housing comprises a passive scrubber.

Aspect 183 provides a method of any one of Aspects 156-182, wherein the air stream housing comprises zeolite, activated carbon, manganese dioxide, or a combination thereof.

Aspect 184 provides a method of any one of Aspects 156-183, wherein the air stream housing comprises a scrubber including activated charcoal, manganese dioxide, or both.

Aspect 185 provides a method of any one of Aspects 156-184, wherein the fan system is one or more tubeaxial fans.

Aspect 186 provides a method of any one of Aspects 156-185, wherein the fan system is one or more fans each having a diameter of 500 mm or less.

Aspect 187 provides a method of any one of Aspects 156-186, wherein the fan system is one or more fans each about 6 W or less, and taken together the fan system is 200 W or less.

Aspect 188 provides a method of any one of Aspects 156-187, wherein the fan system is one or more tubeaxial fans each providing a static pressure of about 0.5 inches H₂O or less, and taken together the fan system provides a static pressure of about 0.5 inches H₂O or less.

Aspect 189 provides a method of any one of Aspects 156-188, wherein the fan system provides a static pressure against filter of about 0.1 inches H₂O or less at a CFM of at least 100 to about 0.5 inches H₂O or less at a CFM of at least 500.

Aspect 190 provides a method of any one of Aspects 156-189, wherein the fan system is two to six tubeaxial fans having a diameter of 150 mm or less.

Aspect 191 provides a method of any one of Aspects 156-190, wherein the far-UV radiation source is a barrier discharge lamp.

Aspect 192 provides a method of any one of Aspects 156-191, wherein the far-UV radiation source is a KrCl excimer lamp.

Aspect 193 provides a method of any one of Aspects 156-192, wherein the far-UV radiation source comprises a bulb having an annular body having an outer surface and defining an internal discharge cavity, wherein the annular body has a major axial dimension and a minor radial dimension, and comprises an electrode in the internal discharge cavity that traverses along the major axial dimension of the annular body.

Aspect 194 provides a method of any one of Aspects 156-193, wherein the far-UV radiation source comprises a lamp housing, a bulb, a reflector, and a window.

Aspect 195 provides a method of any one of Aspects 156-194, wherein the far-UV radiation source comprises an optical filter.

Aspect 196 provides a method of any one of Aspects 156-195, wherein the far-UV radiation source comprises an optical filter that substantially prevents transmittance of one or more wavelength outside of the range between about 200 nm to about 230 nm.

Aspect 197 provides a method of any one of Aspects 156-196, wherein the far-UV radiation source comprises an optical filter that substantially transmits one or more wavelength outside of the range between about 200 nm to about 230 nm.

Aspect 198 provides a method of any one of Aspects 156-197, wherein the far-UV radiation source is integrated with the air stream housing via a fastener, an adhesive, an interlocking part, welding, or being molded together with an exterior surface of the air stream housing.

Aspect 199 provides a method of any one of Aspects 156-198, wherein the far-UV radiation source is integrated with the air stream housing by placement at an air intake or air exhaust of the air stream housing.

Aspect 200 provides a method of any one of Aspects 156-199, wherein the far-UV radiation source is integrated with the air stream housing via a shared stand or mount.

Aspect 201 provides a method of any one of Aspects 156-200, wherein the far-UV radiation source and the air stream unit are integrated together as a single portable system.

Aspect 202 provides a method of any one of Aspects 156-201, wherein the air stream housing is attached to a return or a supply of an HVAC system.

Aspect 203 provides a method of any one of Aspects 156-202, wherein the far-UV radiation source is integrated with the air stream housing via a shared power supply.

Aspect 204 provides a method of any one of Aspects 156-203, wherein the far-UV radiation source is located within 24 inches of the air intake or air exhaust.

Aspect 205 provides a method of any one of Aspects 156-204, wherein the air intake and air exhaust provide one ore more rotary vortices in the human-use space.

Aspect 206 provides a method of any one of Aspects 156-205, wherein the air stream at the air intake and the air exhaust is at least 100 ft³/min.

Aspect 207 provides a method of any one of Aspects 156-206, further comprising collecting spectrophotometric data, irradiance data, or spectral data of one or more far-UV radiation sources, one or more far-UV radiation sources, air quality data, gas content data, sound data, light data, usage data, system status, maintenance-related data, room context data, room occupancy data, or any combination thereof.

Aspect 208 provides a method of any one of Aspects 156-207, further comprising collecting spectrophotometric data, irradiance data, or spectral data of one or more far-UV radiation sources, air quality data, gas content data, sound data, light data, usage data, system status, maintenance-related data, room context data, room occupancy data, or any combination thereof, and adjusting a performance parameter of the far-UV radiation source or air stream unit based on collected data.

Aspect 209 provides a method of any one of Aspects 156-208, further comprising collecting spectrophotometric data, irradiance data, or spectral data of one or more far-UV radiation sources, air quality data, gas content data, sound data, light data, usage data, system status, maintenance-related data, room context data, room occupancy data, or any combination thereof, and adjusting one or more performance parameter of one or more of a filter system, a mechanical filter, or a scrubber.

Aspect 210 provides a method of any one of Aspects 156-209, collecting spectrophotometric data, irradiance data, or spectral data of one or more far-UV radiation sources, air quality data, gas content data, sound data, light data, usage data, system status, maintenance-related data, room context data, room occupancy data, or any combination thereof, and notifying a user of suggested maintenance or changes to a performance parameter.

Aspect 211 provides a method of any one of Aspects 156-210, wherein the air stream housing is configured to provide a diffuse exhaust.

Aspect 212 provides a method of any one of Aspects 156-211, wherein the air exhaust is not suitable for providing an airflow wall.

Aspect 213 provides a method of any one of Aspects 156-212, wherein the air exhaust facilitates scattering of droplets, aerosols, and particles in air.

Aspect 214 provides a method of any one of Aspects 156-213, wherein the far-UV radiation source does not contain a light guide.

Aspect 215 provides a method of any one of Aspects 156-214, wherein a ratio of total fan power/total optical power is about 500-1,000 with an air stream unit configured for a volumetric air flow of at least 500 m³/hr.

Aspect 216 provides a method of any one of Aspects 156-215, wherein a ratio of total optical power/volumetric air flow is about 0.05 mW/(m³/hr) to about 1.00 mW/(m³/hr) with 100 W or less of total fan power, and 150 W or less of total power to the far-UV radiation source and air stream unit.

Aspect 217 provides a method of any one of Aspects 156-216, wherein a ratio of total optical power/total filter surface area is about 0.5 mW/m² to about 10 mW/m² with an air stream unit configured for a volumetric air flow of at least 850 m³/hr and a 100 W or less of total fan power.

Aspect 218 provides a method of any one of Aspects 156-217, which provides a clean air delivery rate (CADR) of at least 300 m³/h.

Aspect 219 provides a method of any one of Aspects 156-218, which provides a volumetric air flow of 350 m³/h.

Aspect 220 provides a method of any one of Aspects 156-219, which provides an air velocity of at least 0.6 m/sec 0.25 m from the air exhaust.

Aspect 221 provides an apparatus, method, or system of any one or Aspects 1-220, which provides or is configured to provide a localized region of air movement at a region irradiated air.

Aspect 222 provides an apparatus, method, or system of any one or Aspects 1-221, which provides or is configured to provide a localized region of high relative velocity air at a region of high relative fluence rate.

Aspect 223 provides an apparatus, method, or system of any one or Aspects 1-221, which provides or is configured to provide a localized region of air having a velocity above the average room air velocity at a region irradiated air.

Aspect 224 provides an apparatus, method, or system of any one or Aspects 1-221, which provides or is configured to provide a localized region of air that has a peak air velocity at a region of irradiated air.

Aspect 225 provides an apparatus, method, or system of any one or Aspects 1-221, which provides a localized region of air having a velocity above the average room air velocity at a region of irradiated air having a fluence rate about or at least 50%, 60%, 70%, 80%, 85%, 90%, 95%, 99%, 99.5%, or 99.9% of the peak fluence rate provided by the far-UV radiation source.

Aspect 226 provides an apparatus, method, or system of any one or Aspects 1-221, which provides a localized region of air having a peak air velocity at a region of irradiated air having a fluence rate about or at least 50%, 60%, 70%, 80%, 85%, 90%, 95%, 99%, 99.5%, or 99.9% of the peak fluence rate provided by the far-UV radiation source.

Aspect 227 provides an apparatus, method, or system of any one or Aspects 1-221, which provides a localized region of air having a velocity above the average room air velocity at a region of irradiated air within about or at least 1, 2, 3, 4, 5, 6, 7, 8, 9, 10, 11, 12, 14, 16, 18, 20, 22, 24, 36, 48, or 60 inches of the far-UV radiation source.

Aspect 228 provides an apparatus, method, or system of any one or Aspects 1-221, which provides a localized region of air having a peak air velocity at a region of irradiated air within about or at least 1, 2, 3, 4, 5, 6, 7, 8, 9, 10, 11, 12, 14, 16, 18, 20, 22, 24, 36, 48, or 60 inches of the far-UV radiation source.

Aspect 229 provides an apparatus, method, or system incorporating any combination or permutation of one or more of the aforementioned features in Examples 1-228, except excluding a filter or scrubber such that the air stream system serves solely to provide an air stream at a localized region of irradiated air.

Aspect 230 provides an apparatus, method, or system incorporating any combination or permutation of one or more of the aforementioned features in Examples 1-229.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. An apparatus for inactivating a pathogen in air, comprising:
an air stream unit comprising an air stream housing defining an air flow chamber, an air intake, and an air exhaust;
a filter system comprising a mechanical filter, a scrubber, or both, disposed in the air flow chamber;
a fan system configured to provide an air stream through the air intake, air flow chamber, and air exhaust; and
a far-UV radiation source that emits radiation comprising one or more wavelengths of about 210 nm to about 230 nm, wherein the far-UV radiation source is integrated with the air stream unit external to the air flow chamber and adjacent to at least one of the air intake or air exhaust, and the far-UV radiation source is positioned to direct radiation away from the air stream housing, filter, and scrubber, if present.

2. The apparatus of claim 1, wherein the air stream housing and filter system is substantially free of direct radiation from the far-UV radiation source.

3. The apparatus of claim 1, wherein the air stream housing is configured to displace irradiated air proximate to the far-UV radiation source.

4. The apparatus of claim 1, wherein the air intake is adjacent to the far-UV radiation source and the air stream housing is configured to capture irradiated air proximate to the far-UV radiation source.

5. The apparatus of claim 1, wherein the air stream housing is configured to capture irradiated air and displace it in a direction substantially opposite to a direction of the emitted radiation.

6. The apparatus of claim 1, wherein the air exhaust is adjacent to the far-UV radiation source and the air stream housing is configured to exhaust the air stream into a space irradiated by the far-UV radiation source.

7. The apparatus of claim 1, which comprises one or more mechanical filters, and the one or more mechanical filters have a penetration of 5% or greater at 0.3 microns, each individually, taken together, or both.

8. The apparatus of claim 1, which comprises one or more mechanical filters, and the one or more mechanical filters have a penetration of 40% or greater at 0.3 microns, each individually, taken together, or both.

9. The apparatus of claim 1, which comprises one or more mechanical filters, and the one or more mechanical filters are not HEPA filters and do not satisfy HEPA standard ASME AG-1, when considered each individually, taken together, or both.

10. The apparatus of claim 1, which comprises one or more mechanical filters, and the one or more mechanical filters have a flow rate of at least 50 CFM at a pressure drop of 0.25 or less, each individually, taken together, or both.

11. The apparatus of claim 1, wherein the fan system is one or more tubeaxial fans each providing a static pressure of about 0.5 inches H₂O or less, and taken together the fan system provides a static pressure of about 0.5 inches H₂O or less.

12. The apparatus of claim 1, wherein the fan system is two to six tubeaxial fans having a diameter of 150 mm or less.

13. The apparatus of claim 1, wherein the far-UV radiation source is a KrCl excimer lamp.

14. The apparatus of claim 1, wherein the far-UV radiation source is integrated with the air stream housing via a fastener, an adhesive, an interlocking part, welding, or being molded together with an exterior surface of the air stream housing.

15. The apparatus of claim 1, which is configured for attaching the air stream housing to a return or a supply of an HVAC system.

16. The apparatus of claim 1, wherein the far-UV radiation source is located within 24 inches of the air intake or air exhaust.

17. The apparatus of claim 1, configured to provide a localized region of high relative velocity air at a region of high relative UV fluence rate.

18. The apparatus of claim 1, configured to provide a localized region of air having a velocity above an average room air velocity at a region of irradiated air within 24 inches of the far-UV radiation source.

19. The apparatus of claim 1, configured to provide a localized region of air having a velocity above an average room air velocity at a region of irradiated air having a fluence rate at least 90% of a maximum fluence rate provided by the far-UV radiation source.

20. A system for inactivating a pathogen in air, comprising:
a far-UV radiation source configured for irradiating air of a human-use space, wherein the far-UV radiation source emits radiation comprising one or more wavelengths of about 210 nm to about 230 nm;
an air stream unit comprising an air stream housing that defines an air flow chamber, an air intake, and an air exhaust;
a fan system configured to displace irradiated air proximate to the radiation source and provide an air stream through the air intake, air flow chamber, and air exhaust; and
a filter disposed in the air flow chamber configured for removing particles from the air stream, a scrubber disposed in the air flow chamber configured for removing reactive agents from the air stream, or both;
wherein the far-UV radiation source is integrated with the air stream unit external to the air flow chamber and adjacent to at least one of the air intake or air exhaust, and the far-UV radiation source is positioned such that substantially all radiation is directed away from the air stream housing.
